(19) United States
(12) Patent Application Publication
(10) Pub. No.: US 2014/0012228 A1
(43) Pub. Date: Jan. 9, 2014

(54) METHODS, DEVICES AND SYSTEMS FOR ACCESSING A HOLLOW ORGAN

(71) Applicant: Promed, Inc., Santa Clara, CA (US)

(72) Inventors: Ronald J. JABBA, Redwood City, CA (US); Anthony J. PANTAGES, San Jose, CA (US); Mark REISMAN, Seattle, WA (US)

(21) Appl. No.: 13/923,270

(22) Filed: Jun. 20, 2013

Related U.S. Application Data
(63) Continuation of application No. 13/634,180, filed on Aug. 7, 2013.
(60) Provisional application No. 61/310,445, filed on Mar. 4, 2010.

(51) Int. Cl. A61B 17/34

(52) U.S. Cl. CPC A61B 17/3415
USPC 604/506; 606/185

(57) ABSTRACT

The methods, devices and systems described herein relate to accessing a hollow organ with increased accuracy and safety. In one example, they relate to transseptal crossings of the atrial septal wall, preferably from the right atrium into the left atrium. Numerous different devices are described for performing access methods, each of which can be used with a piercing stylet that transitions to an atraumatic configuration after piercing the intervening tissue.

### FIELD

The methods, devices and systems described herein relate to accessing a hollow organ. In one example, they relate to improved and safer delivery devices and methods for performing a transseptal crossing into the left atrium from the right atrium across an atrial septal wall.

### BACKGROUND

By nature of their location, accessing and treating internal tissues and/organs is inherently difficult. Invasive surgery introduces a high level of risk that can result in serious complications for the patient. Access to the organ remotely with a catheter or equivalent device is less risky, but a desired treatment is made more difficult given the limited physical abilities of the catheter. This difficultly is compounded when the targeted treatment site is found in or near a vital organ, such as the heart.

A human heart includes a right ventricle, a right atrium, a left ventricle and a left atrium. The atria and ventricles are each separated by a septal wall. The fossa ovalis is a thin, distensible portion of the atrial septal wall. Because the right atrium is in fluid communication with the superior vena cava and the inferior vena cava, it is relatively accessible intravenously. Access to the left atrium (and ventricles) is more difficult and typically requires traversing the aortic arch and aortic valve. Therefore, access to the left atrium is commonly obtained using a transseptal procedure, which usually requires puncturing the atrial septal wall.

The transseptal approach for left atrial or ventricular access has been known in the art for some time and can be used in a variety of procedures such as percutaneous balloon mitral valvuloplasty, antegrade percutaneous aortic valvuloplasty as well as catheter ablation of arrhythmias arising from the left atrium or utilizing left sided bypass tracts. The transseptal approach is typically used to cross from the right atrium to the left atrium through the fossa ovalis. In a transseptal procedure, a needle and catheter are generally used to puncture the atrial septal wall to the fossa ovalis.

In a typically transseptal procedure, a guidewire is first inserted through the right femoral vein and advanced to the superior vena cava. Sometimes, a sheath is placed over a dilator that is advanced over the guidewire into the superior vena cava. The guidewire is then removed and a puncture device, such as a Brockenbrough needle, is advanced up to the dilator tip. The apparatus is dragged down, pushed up, or a combination of both as necessary into the right atrium, along the septum. When the dilator tip is positioned adjacent the fossa ovalis (sometimes determined under ultrasound, fluoroscopic, or other guidance), the needle is then advanced forward so that it extends past the dilator tip, through the fossa ovalis into the left atrium. The dilator and sheath may then be advanced through the fossa ovalis over the needle. The dilator and needle are then removed, leaving the sheath in place in the left atrium. Thereafter, a guidewire or catheter may be inserted into the left atrium (through the sheath) in order to perform the desired procedure. Additional techniques may be used to determine position such as biplane fluoroscopy, pressure manometry, contrast infusion, transesophageal or intracardiac echocardiography.

Current techniques for transseptal approach come with a relatively high degree of risk that the transseptal puncture device will damage tissue near the septal wall, such as the aorta, the coronary sinus or the far wall of the atrium. Accordingly, improved systems and methods for performing a transseptal crossing within the heart are needed.

### SUMMARY

Improved systems, devices and methods for accessing a hollow organ, such as accessing the left atrium from the right atrium using a transseptal crossing, are provided herein by the way of exemplary embodiments. These embodiments are examples only and are not intended to limit the invention.

In one exemplary embodiment, a method for accessing a hollow organ, such as a left atrium, is provided, including: accessing the organ percutaneously; advancing a guidewire into the organ; advancing an elongate member having a delivery device along the guidewire into the organ, or for example into the right atrium for transseptal access to a left atrium; adjusting an orientation of the distal end of the delivery device from a first position to a variable second position, wherein the second position is in a desired orientation with respect to a target portion of the organ, such as an atrial septal wall, and is deflected away from the normal bias or a longitudinal axis of a proximal end of the elongate member; and using the delivery device to access and/or deliver other medical instruments to the organ, for performing one or more desired medical procedures on (or by way of) the organ. Optionally, another adjustment may be made to a distal end of an elongate member of the access system. Typically, these procedures are performed using imaging for guidance. In one embodiment, the method is used to create a transseptal puncture to facilitate a later medical treatment, such as ablation of abnormal tissue.

In one exemplary embodiment a system for accessing a left atrium is provided, including: an elongate member with a distal end and at least one inner lumen; adjustment device configured to allow the orientation of the distal end of the elongate member to change from a first position to a variable second position, wherein the second position is in a desired orientation with respect to a septal wall and is deflected away from a longitudinal axis of a proximal end of the elongate member; and a puncture device housed at least partially within the inner lumen of the elongate member in a first configuration, wherein the puncture device optionally assumes an atraumatic second configuration upon advancement from the elongate member.

In another example embodiment, a delivery device is configured to be housed at least partially within the inner lumen of the elongate member in a first position; and the delivery device is advanceable through a first open region of the elongate member to a variable second position in a desired orientation to a septal wall, wherein the variable second position is external to the elongate member and deflected away from the elongate member; and a puncture device housed at least partially within an inner lumen of the delivery device in a first configuration, wherein the puncture device assumes an atraumatic second configuration upon advancement from the elongate member.

The foregoing is not an exhaustive list of embodiments. Other systems, methods, features and advantages of the invention will be or will, become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1A is a exterior/interior view depicting an example human heart with a portion of the inferior vena cava and the superior vena cava connected thereto.

FIG. 1B is a cross-sectional view depicting an example atrial septal wall.

FIG. 2A is a perspective view depicting an exemplary embodiment of an access system.

FIGS. 2B-D are cross-sectional views depicting additional exemplary embodiments of an access system.

FIGS. 2B-F are perspective views depicting an exemplary embodiment of system 100 in use to access the left atrium.

FIG. 2G is a longitudinal cross-sectional view depicting an exemplary embodiment of system 100 with a pull wire.

FIG. 2H is a side view depicting an exemplary embodiment of a piercing stylet.

FIG. 2I is an end-on view depicting another exemplary embodiment of a piercing stylet.

FIG. 3A is a perspective view depicting an exemplary embodiment of system 100 in use to access the left atrium.

FIG. 3B is a longitudinal cross-sectional view depicting another exemplary embodiment of an access system.

FIGS. 4A-B are perspective views depicting additional exemplary embodiments of an access system.

FIGS. 5A-I are perspective views depicting additional exemplary embodiments of an access system at various stages of use.

FIG. 5J is a perspective view depicting an exemplary embodiment of an extension member.

FIG. 5K is a cross-sectional view taken along line 5K-5K of FIG. 5J.

FIG. 5L is a cross-sectional view taken along line 5L-5L of FIG. 5J.

FIGS. 5M-N are perspective views depicting additional exemplary embodiments of an extension member.

FIG. 6 is partial cutaway view of a heart with various regions of the atrial septal wall identified.

FIGS. 7A-E are top down views depleting additional exemplary embodiments of an access system.

FIG. 8 is top down view depicting an exemplary embodiment of a proximal controller.

### DETAILED DESCRIPTION

Devices, systems and methods for accessing an internal hollow organ are described herein. For ease of discussion, these devices, systems and methods will be described with reference to transseptal access of a left atrium. However, it should be understood that these devices, systems and methods can be used to access a variety of hollow organs and to assist in delivery of a variety of medical instruments. In addition, one of skill in the art will readily recognize that the devices, systems and methods can be similarly applied to access the right atrium from the left atrium.

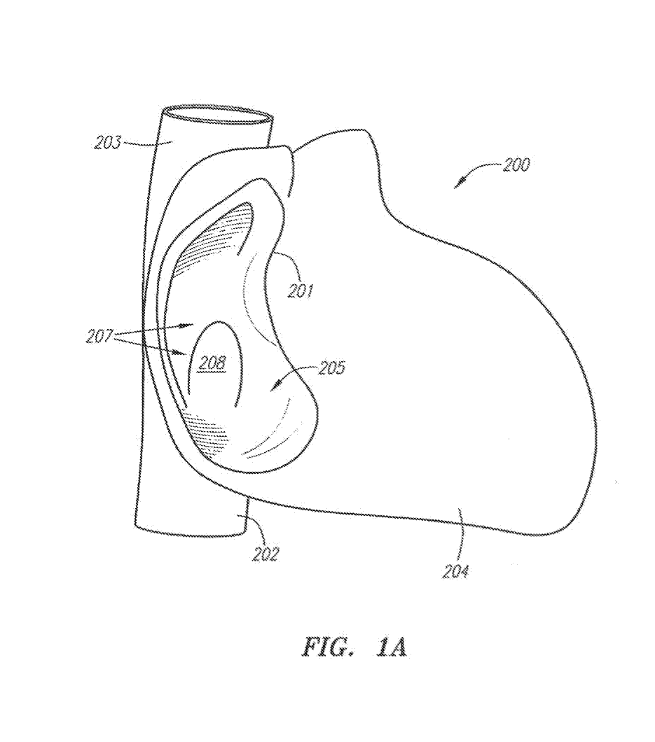

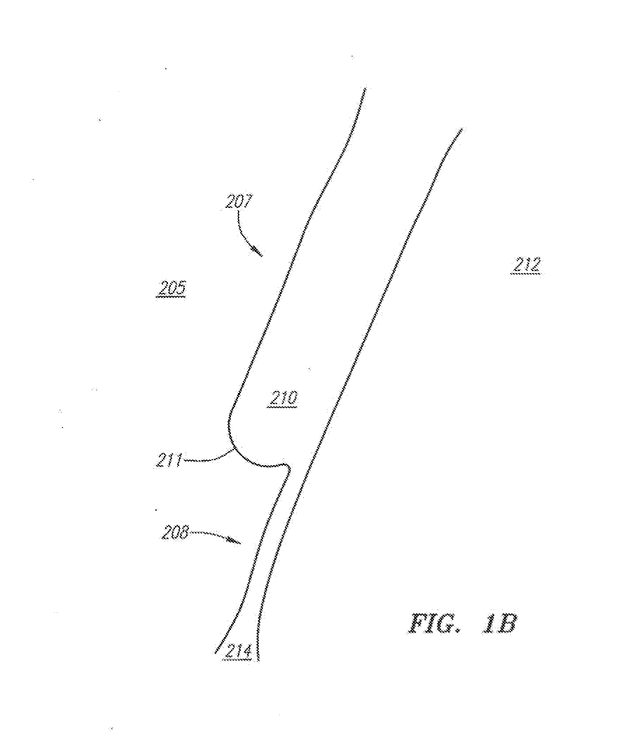

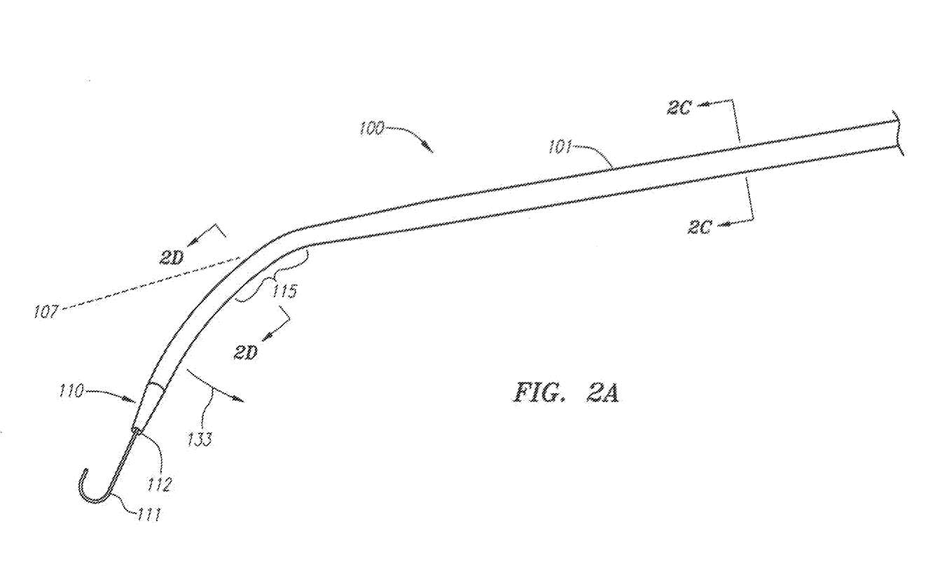

To facilitate description of the many alternative embodiments of access system 100, the anatomical structure of an example human heart will be described in brief. FIG. 1A is an exterior/interior view depicting an example human heart 200 with a portion of the inferior vena cava (IVC) 202 and the superior vena cava (SVC) 203 connected thereto. Outer tissue surface 204 of heart 200 is shown along with the interior of right atrium 205 via cutaway portion 201. Depicted within tight atrium 205 is septal wall 207, which separates the right atrium 205 from the left atrium, located on the opposite side (not shown). Also depicted is fossa ovalis 208, which is a region of septal wall 207 having tissue that is relatively thinner than the surrounding tissue.

FIG. 1B is an enlarged cross-sectional view depicting an example septal wall 207. Before birth, the atrial septal wall is composed of two flaps, septum primum and a septum secundum, with a tunnel, or patent foramen ovale, existing there between. The foramen ovale allows blood to pass directly between the left and right atria in order to bypass the lungs in utero. After birth, the primum and secundum typically fuse together to seal the foramen ovale (failure to seal results in a patent foramen ovale condition, which can persist into adulthood).

FIG. 1B shows atrial septal wall 207 between right atrium 205 and left atrium 212. Septal wall 207 is shown here after normal fusing of septum secundum 210 and septum primum 214. The portion of septal wall 207 corresponding to septum secundum 210 is typically relatively thicker than the inferior septum primum 214, which includes fossa ovalis region 208 that is much thinner and more distensible. The inferior edge of the septum secundum 210 is referred to as limbus 211. After fusing, limbus 211 typically remains as an outcropping extending from the surface of septal wall 207.

It is desirable to gain access from one atrium to the other by passing a device through the atrial septal wall 207, i.e., a transseptal procedure. As will be described in more detail below, the transseptal procedure preferably includes inserting access system 100 into the vasculature of a patient and advancing an elongate body member 101 through the vasculature to inferior vena cava 202 (e.g., over a guidewire), from which access to right atrium 205 can be obtained. Once properly positioned within right atrium 205, system 100 can be used to deliver puncture device 103 to septal wall 207.

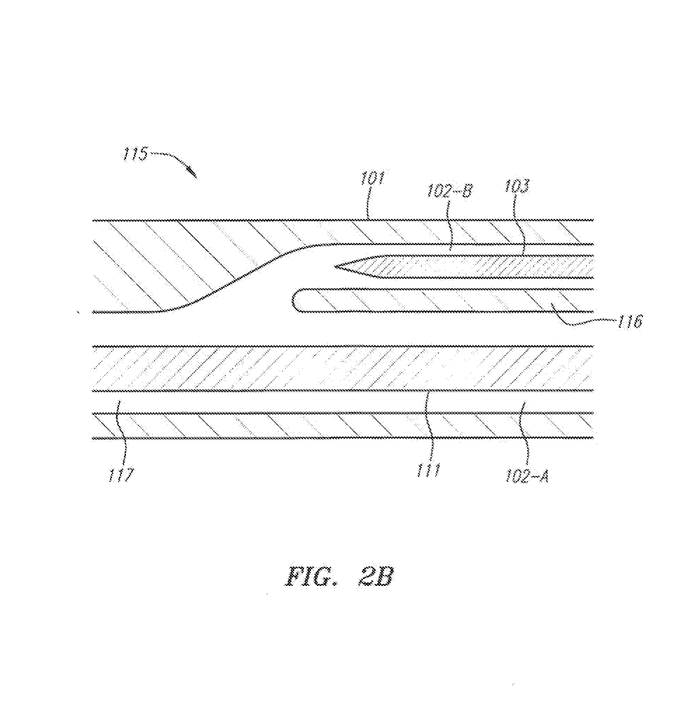

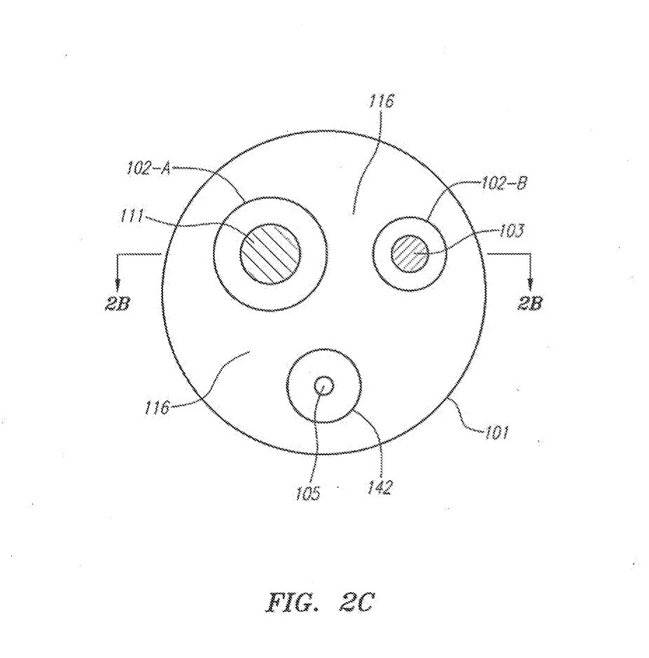

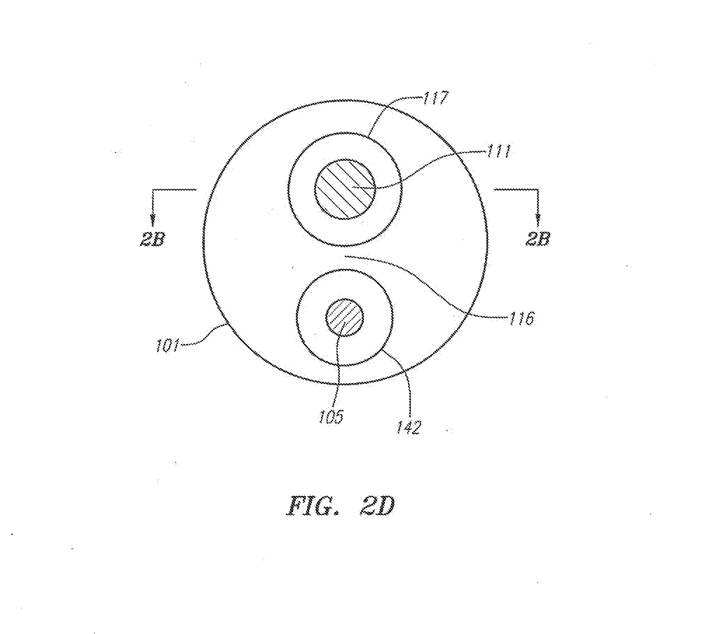

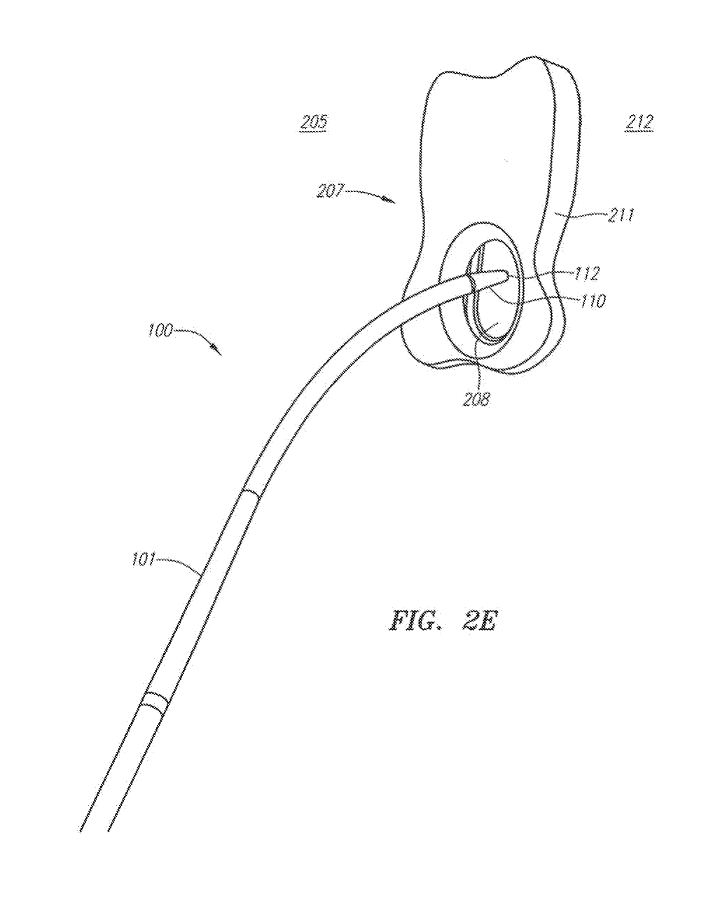

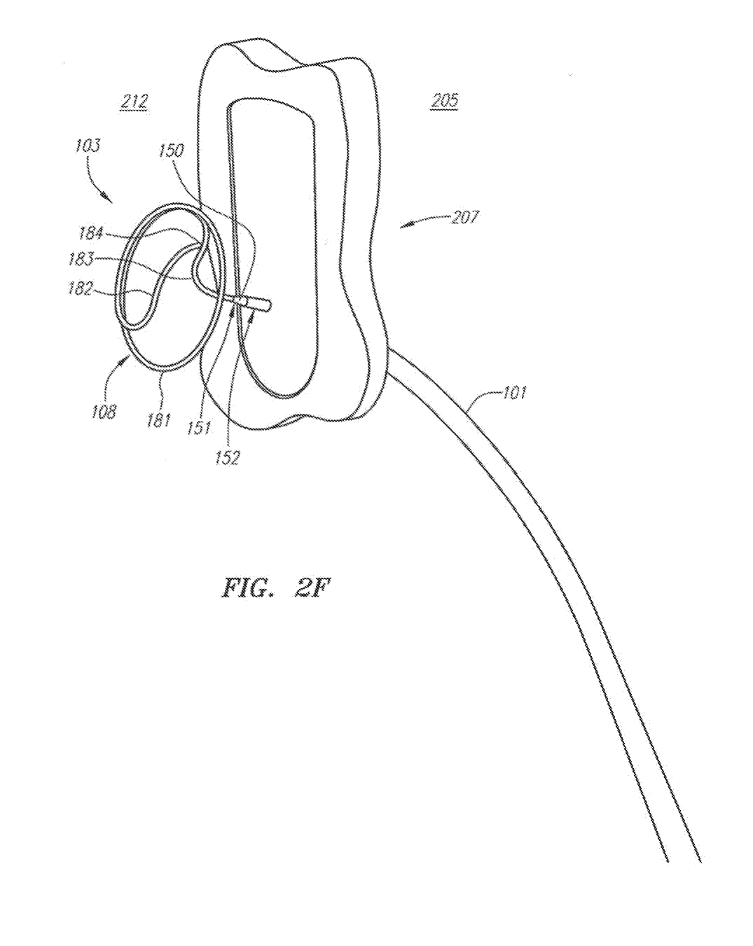

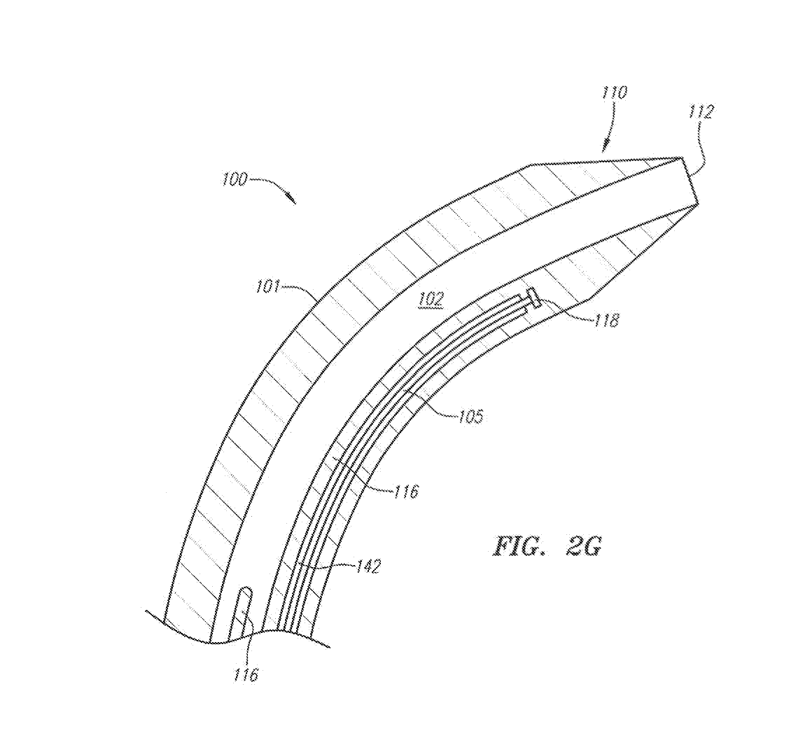

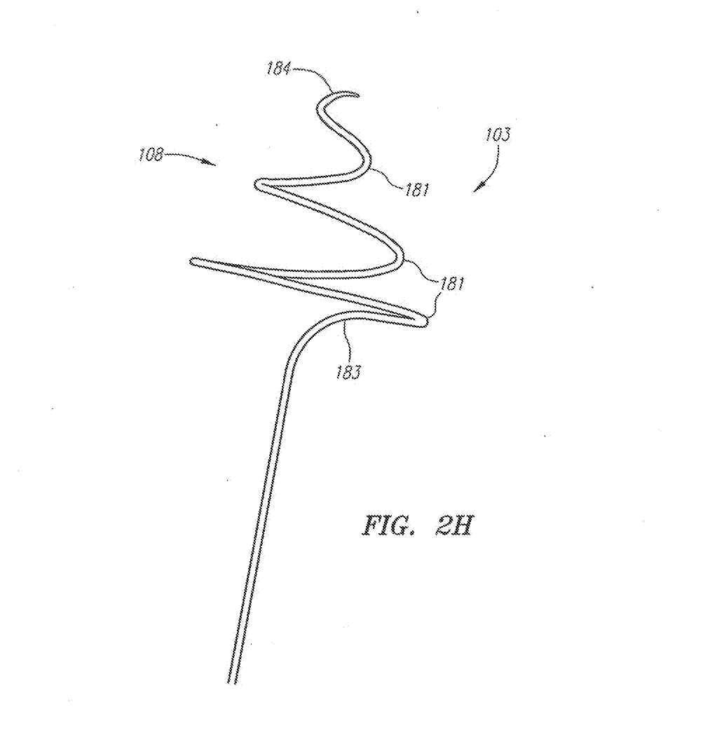

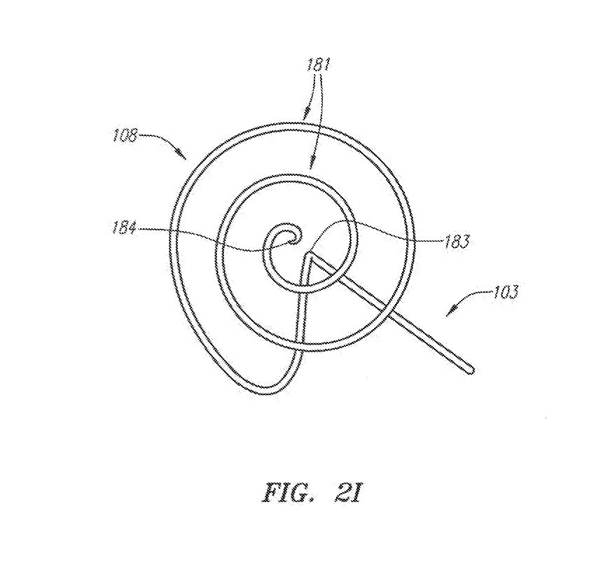

FIGS. 2A-I depict an exemplary embodiment of access system 100, including elongate body member 101, which has a longitudinal axis 107, a distal end 112, and one or more lumens 102 (not shown), each of which can be configured for receiving one or more different medical devices. FIG. 2A is a perspective view showing guidewire 111 exiting from distal end 112, FIG. 2B is a longitudinal cross-sectional view of region 115, taken along line 2B-2B of FIGS. 2C and 2D, FIG. 2C is a transverse cross-sectional view taken along line 2C-2C of FIG. 2A, and FIG. 2D is a transverse cross-sectional view taken along line 2D-2D of FIG. 2A. FIGS. 2E-F are perspective views depicting system 100 at various stages of use, while FIG. 2G is a longitudinal cross-sectional view of system 100 showing adjustment device 105. FIGS. 2H-I are perspective views of an exemplary embodiment of puncture device 130. The description with respect to FIGS. 2A-I can be likewise applied to all embodiments described herein.

Elongate member 101 can be any of a variety of devices capable of insertion into a patient's (human, or animal) vasculature, including but not limited to a hollow tubular member, such as a catheter. Access system 100 can also optionally include an adjustment device 105 (see FIG. 2G) configured to position the distal end 112 of elongate member 101 into a desired orientation. Typically, at least a portion of elongate member 101 is made of flexible materials, capable of deforming as necessary for travel within the patient's body and/or in response to an adjustment device 105, such as the pull wire depicted in FIG. 2G. Elongate member 101 can be biased towards a substantially straight state (e.g., lying generally along axis 107 as depicted in FIG. 2A) or member 101 can be biased to curve (e.g., such that the at-rest state of elongate member is as shown in FIG. 2A). In this embodiment, distal end 112 is curved 45 degrees from longitudinal axis 107 when there is no tension on pull wire 105 (i.e., when member 101 is at-rest). This can be accomplished by heat-treating member 101 with distal end 112 curved at the 45 degree angle. In this embodiment, the pre-biased distal end can deflect an additional 15 degrees (or more) in direction 133 when proximal tension is placed on pull wire 105. Of course, these are only example angles of deflections and one or ordinary skill in the art can configure the device to have or operate across larger or smaller ranges of deflection.

Any portion of access system 100 can optionally include an inter lumen exchange system 115. Inter lumen exchange system 115 allows the catheter, such as elongate member 101 (or delivery device 104, which is described later) to house multiple elongate devices. As mentioned, the one or more lumens 102 and 142 can. be configured to house a variety of devices, such as puncture device 103, guidewire 111, pull wire 105, or other medical devices. FIGS. 2B-D depict guidewire 111 within lumen 102-A and 117, puncture device 103 within lumen 102-B, and pull wire 105 within lumen 142 (FIGS. 2G-D only).

FIG. 2B depicts a longitudinal cross-sectional view of an example embodiment of elongate member 101 having an inter lumen exchange system 115. In this cross-sectional plane (see line 2B-2B of FIG. 2D), two lumens 102 are present along the length of the elongate member and separated by dividing wall 116. The two lumens 102 integrate into one common lumen 117, preferably near distal end 112 of elongate member 101. (In one exemplary embodiment, the transition to one common lumen 117 occurs approximately 5 centimeters from distal end 112, although it should be noted that this transition point can occur at any desired location along the length of the catheter.)

Here, lumen 102-A is aligned with common lumen 117 and lumen 102-B is offset from common lumen 117. This can facilitate the loading of components into member 101. For instance, loading the proximal end of a guidewire through distal end 112 of member 101 and into lumen 102-I is made easier by aligning lumens 117 and 102-A and making it more difficult to accidentally load the guidewire proximal end into lumen 102-B, which may house a different component such as piercing structure 103.

FIG. 2C depicts a transverse cross-sectional view of elongate member 101 located proximal to the three-to-two lumen transition of inter lumen exchange system 115. Each of the three lumens (two lumens 102 and one lumen 142) are separated by dividing walls 116. FIG. 2D depicts a transverse cross-sectional view of elongate member 101 after inter lumen exchange system 115. Here, one common lumen 117 is present separated from lumen 142 by dividing wall 16. An elongate device extended from the access system can be partially retracted from lumen 117 into one of lumens 102, while an elongate device in a separate lumen 102 can then be advanced through common lumen 117. Thus, the device that is retracted does not need to be fully retracted from the entire system 100 to exchange it for another. Although this configuration shows lumen 102-A housing a guidewire 111 and lumen 102-B housing puncture device 103, it should be understood that each lumen can be configured to carry any of the elongate components described herein.

Any variety of guidewires known in the art may be used, but a J-tip guidewire, such as a 0.035″ or 0.038″ is presently preferred. This embodiment also includes an optional deployable dilator 131. The distal region 110 of elongate member 101 includes distal end 112 and can be tapered to act as a dilator, and can be used alone or with an additional dilator extendable from within. Some embodiments may also facilitate dye injection, for instance, through open distal end 112 or another distal dye injection port communicating with a lumen 102, to aid in determining positioning. It should be noted that any number of lumens 102 can be provided and any type of treatment device can be received within each lumen 102, including, but not limited to a valvuloplasty device (e.g., balloons, etc.), a radio frequency (RF) ablation device, an imaging device, and the like. Elongate member 101 can also be used as a component within a larger catheter sheath.

In FIGS. 2C-D, another inner lumen 142 is configured to house pull wire 105 and extends from the proximal end of elongate member 101 until a position just proximal to distal end 112, where the distal tip of pull wire 105 can be anchored. Lumen 142 can be used to accommodate adjustment device 105 and, if so, can be positioned along the inside of any pre-biased curve in member 101.

As seen in FIG. 2G, pull wire 105 can be fixed to the distal end of lumen 142 with an anchor 118 embedded within (or otherwise connected to) the wall of member 101. Pull wire 105 allows distal end 112 of elongate member 101 to be deflected from a first position (e.g., the position to which elongate member 101 is normally biased) to a variable second position. (Components other than member 101 and pull wire 105 have been omitted from FIG. 2G for clarity.) The second position is typically deflected away from (or further away from) longitudinal axis 107 (e.g., as determined at the portion of the elongate member 101 located immediately proximal to the bend). The variable second position is selected to facilitate the desired orientation of elongate member 101 with respect to septal wall 207. Access system 100 can be configured to bend, or deflect, in any direction (north, south, east, west) from the first position to the desired second position. Accordingly, any number of pull wires 105 can be included and positioned within elongate member 101 to allow for this multi-directional adjustment. For instance, in another example embodiment, four pull wires 105 are coupled to elongate member 101 at ninety degree intervals about the periphery of the outer wall of member 101. Furthermore, variations in the flexibility of elongate member 101 (or device 104, described below) can be employed to facilitate bending at the appropriate location in the catheter. For instance, a thinner (and hence more flexible) wall can be used at the location where the bend is intended to occur. More flexible materials could also (or alternatively) be used at that location to achieve the same effect. Also, the region where the bend occurs can be reinforced with coils or braid to improve kink resistance during bending.

An exemplary method for transseptal left atrium access using the exemplary embodiments described with respect to FIGS. 2A-G is provided. For this and all method embodiments described herein, steps are described generally sequentially (e.g., using terms such as “then” and “next”). This is done for ease of discussion only and one of skill in the art will readily recognize that it can be possible to perform the steps in other orders. Furthermore, although these method examples are described with a detail number of steps, not all recited steps are required to be practiced.

First, a patient's femoral vein (or other vasculature capable of being used to access right atrium 205) is accessed. Optionally, an introducer sheath is inserted into the selected vein. Next, a guidewire can be advanced through the sheath, and navigated through the patient's vascular system into the right atrium 205, preferably from the IVC through the right atrium and into the SVC. The guidewire can be used to guide additional components along the same path into right atrium 205.

More specifically, in one example, a needle is inserted into the femoral vein and a guidewire is advanced through the needle into that vein. The needle can then be removed and an access (or introducer) sheath can be routed over the guidewire. The access guidewire is preferably removed prior to inserting the guidewire used to access the heart. Typically, access to the heart is achieved with a J-tip guidewire, such as a 0.035″/0.038″ guidewire, which can be routed through the patient's vasculature into inferior vena cava 202 and then right atrium 205. The distal tip of this guidewire can be placed in the superior vena cava 203. The distal position of this guidewire can then be confirmed using imaging techniques. The confirmation of the positioning of the guidewire, as well as any desired portion of access system 100 throughout the procedure, can be accomplished from one or more of a variety of imaging techniques such as biplane fluoroscopy, pressure manometry, contrast infusion, transesophageal, intracardiac echocardiography, intravascular ultrasound, external ultrasound, or other imaging techniques. Any desired portion of access system 100 may also be composed of radiopaque materials to aid in determining positioning with fluoroscopy.

A proximal end of the guidewire can then be loaded into distal end 112 of elongate member 101. Elongate member 101 can then advanced through the introducer sheath and along the guidewire into the right atrium 205 or superior vena cava 203. The distal position of elongate member 101 can then be confirmed (again, using fluoroscopy or other imaging techniques). The guidewire can then be retracted at least partially into distal end 112 of elongate member 101, and preferably at about five (5) centimeters (cm) in from the distal end 112 of elongate member 101.

In an exemplary embodiment, the pre-biased distal end 112 is used in positioning with respect to the limbus 211. Alternatively, in embodiments without a pre-biased distal end 112, pull wire 105 can be used to introduce an amount of deflection into elongate member 101 to aid in positioning with respect to limbus 211. Elongate member 101 can then be retracted and adjusted as necessary so that distal end 112 is just inferior of the limbus 211. This can be accomplished using a “feel” technique where the physician (or other medical professional) retracts member 101 along septal wall 207 and uses tactile feedback to determine when distal end 112 passes over (or snaps over) limbus 211 (or alternatively, the physician can advance distal end 112 until it hits limbus 211 and stops further advancement, or the physician can use a combination of both advancement and retraction).

The deflection of distal end 112 of elongate member 101 can then be further deflected (if needed) using pull wire 105, for example, to achieve the desired orientation with respect to fossa ovalis 208. FIG. 2E is a perspective view depicting elongate member 101 in an example desired orientation against fossa ovalis 208. A preferable amount of deflection can be one that allows the distal region of member 101 to align generally perpendicular to septal wall or within about plus or minus 45 degrees of a normal axis to septal wall 207. Other offset angles can be used if desired. Next, the position of distal end 112 can be confirmed, using the aforementioned imaging techniques, to be in a desired orientation with respect to septal wall 207. The orientation of elongate member 101 can be repeatedly adjusted, with or without imaging guidance, as many times as necessary until the desired orientation is confirmed. The desired orientation preferably has the desired degree of deflection in the tip of member 101.

If necessary, preferably continuing under imaging guidance, elongate member 101 can be rotated axially to assess the distention (or amount of dimpling or tenting) of the fossa ovalis 208 by distal end 112. Distension of the desired tissue is achieved by applying a force on the proximal end of access system 100. The degree of curvature in distal end 112, including its pre-bias and that introduced using pull wire 105, will impact the degree of distension. Generally, proper positioning of elongate member 101 is confirmed when the relative preferred amount of dimpling is achieved. A hole can then be created in septal wall 207 using puncture device 103. Preferably, puncture device 103 is an elongate piercing stylet that can be advanced through septal tissue, as depicted in FIG. 2F. As shown, puncture device 103 can assume an atraumatic shape in distal portion 108 after puncturing the patient's tissue (this is described in more detail below). The deployed position of puncture device 103 can be confirmed using imaging techniques. The physician can then perform a left atrial pressure measurement to confirm that access system 100 is positioned in the left atrium 212.

If distal region 110 of elongate member 101 (or device 104, described below) is tapered, it can be advanced into the hole in septal wall 207 to aid in dilating the hole. A separate elongate dilator 131 can also (or alternatively) be advanced from distal end 112 and through the septal opening to enlarge the hole. The deflected atraumatic distal portion 108 acts as a stop to further distal movement of the dilator or any other member advanced through the opening over puncture device 103. This acts to prevent inadvertent injury that would be caused should the dilator tip contact other structures in the left atrium (e.g., left atrial far wall, aorta, etc.). If desired, the atraumatic distal portion 108 can be retracted up against the septum primum prior to advancing dilator 131 (or other dilating portion or distal end) through the septal tissue. This acts to hold the distensible fossa ovalis 208 in position while the dilator widens the initial puncture site.

Next, puncture device 103 can be withdrawn, either partially inside or fully from elongate member 101. Finally, the guidewire can be advanced into left atrium 212. When the guidewire is in a desired position as confirmed using imaging techniques, pull wire 105 may be advanced, relaxing the tension and allowing elongate member 101 to adjust back toward its initial orientation. Access system 100 can then be retracted leaving the guidewire in left atrium 212.

FIG. 2F depicts an example embodiment of puncture device 103 implemented as a piercing stylet 103. Stylet 103 is shown here in with atraumatic distal portion 108 after deployment from elongate member 101, but this embodiment of stylet 103 can be used with all embodiments of system 100 described herein. Stylet 103 is housed in a generally elongate, straight configuration while within system 100. As stylet 103 is advanced from within a component (e.g., catheter, dilator, delivery device) of system 100, the sharp distal tip 184 exits in the generally elongate, straight configuration and pierces the septal tissue in the septal wall. As stylet 103 continues to advance past the septal wall and into the left atrium, it begins to assume the atraumatic configuration. In the atraumatic configuration, stylet 103 is transverse to its initial axis at section 183, assumes one or more loops at annular portion 181, which lies transverse, preferably perpendicular, to the longitudinal axis of the proximally located undeflected stylet. Puncture device 103 then includes a wave-like portion 182, after annular portion 181 and before sharp distal tip 184. Other configurations for atraumatic distal portion. 108 can be used, such as a G-like atraumatic shape, one or more loops transverse to a primary axis of the puncture device, a plurality of loops that can act as an atraumatic compression spring, spiraled coil, circular, rectangular, or have a pattern such as a petal and the like. The shape can be generally planar, such as the two-dimensional configuration depleted in FIG. 2F, or the shape can be three-dimensional (3-D), such as the helical coil configuration depicted in the side view and end-on view of FIGS. 2H-I, respectively. When, advanced into a left heart structure such as the far left atrial wall, this 3-D shape can collapse into a near two-dimensional (2-D) planar shape preventing the tip of the dilator from causing errant punctures (e.g., in the aorta or left atrial far wall, etc).

Puncture device 103 may also include a retractable cover that, when retracted, allows the puncture device to assume its atraumatic shape. Puncture device 103 may also include an advancement control, such as a ratcheting mechanism. The ratcheting mechanism could be used to facilitate a controlled advancement or retraction of the puncture device 103. Proximal controller (described below) may be configured to control the advancement control of the puncture device, including through the ratcheting mechanism. In some embodiments, puncture device 103 may also be spring loaded, for deployment, retraction, or both.

The puncture device 103 or a portion thereof may comprise a flexible metal, a flexible polymer, or a combination thereof. A presently preferred flexible metal for the distal portion of the puncture device is NITINOL. NITINOL allows the puncture device to be deformed to fit within a flexible lumen and to change shape as necessary while carried within the elongate member (or delivery device) through the patient's vasculature, and then to transition to a previously set atraumatic shape upon puncturing a patient's tissue.

When configured as a piercing stylet, puncture device 103 can also include a piercing stylet assembly. The piercing stylet assembly can be housed, e.g., within one of the lumens of the dilator assembly. Although described here as an assembly, one can also implement the stylet as a monolithic device (e.g., with sections 108, 151 and 152 all being formed in one piece of material).

The piercing stylet has a relatively sharp distal end sufficient to pierce the intra-atrial septae tissue layers (septum primum and septum secundum). The piercing distal end can be constructed from Nitinol wire or tube that has been sharpened. The atraumatic section 108 is distal to a tapered junction element 151 that allows a transition between diameters of the relatively narrow distal section and the relatively wider proximal section 152. The reduced profile distal section of the piercing stylet as well as the piercing distal tip 184 is heat treated to a preferred shape.

Tapered section (or junction element) 151 is connected to a tubular proximal section 152 that is housed within the catheter body and exists through the proximal controller handle. The outside diameter of proximal section 152 is approximately the same as the internal diameter of the common lumen 117 section within the dilator (or elongate member, delivery device or other member from which stylet 103 is deployed). These matched profiles create a near interference fit between stylet 103 and the surrounding assembly. Tapered section 151 possesses sufficient cross-sectional lumen space to enable a left atrial pressure measurement to be made by the user from the handle region. For example, tapered section 151 could possess a series of small holes 150 (e.g. circular, crescent) located circumferentially around section 150 that enables the user to measure left atrial pressure as shown in FIG. 2F. It should be noted that at this point in the procedure the distal end of member 101 is preferably located at the intra-atrial septum and piercing stylet 103 is located in the left atrium.

Proximal section 152 of piercing stylet 103 can be constructed from a wire or a tube that is metal or plastic. If a tubular section is used, left atrial pressure can be measured from within the tube. If a wire is used, the blood pressure measurement is based on flow between the outside of the wire and the dilator lumen clearance.

After the pressure measurement is made, piercing stylet 103 can be retracted back into the double lumen section 115. Image guidance such as fluoroscopy can be used to facilitate the retraction of piercing stylet 103.

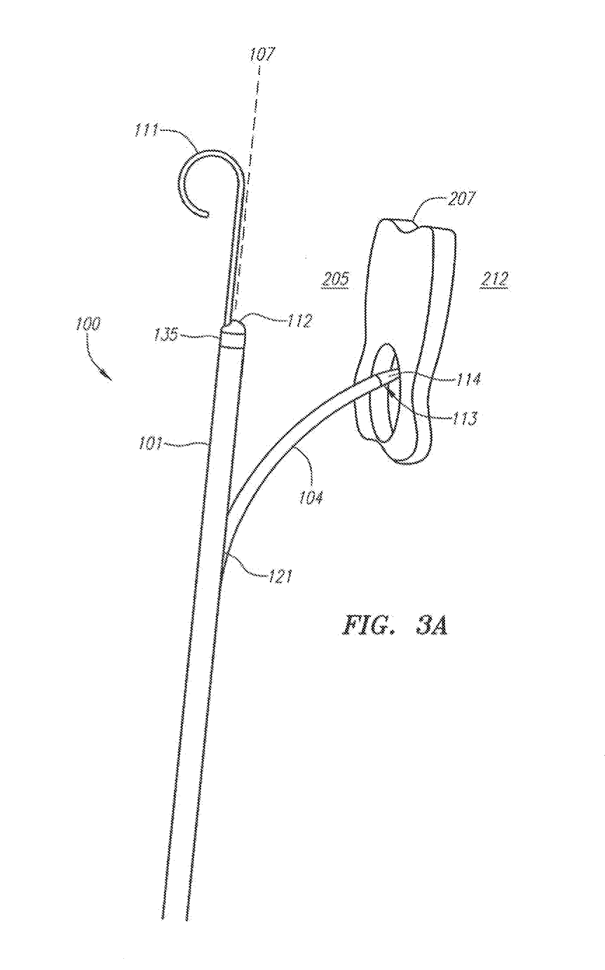

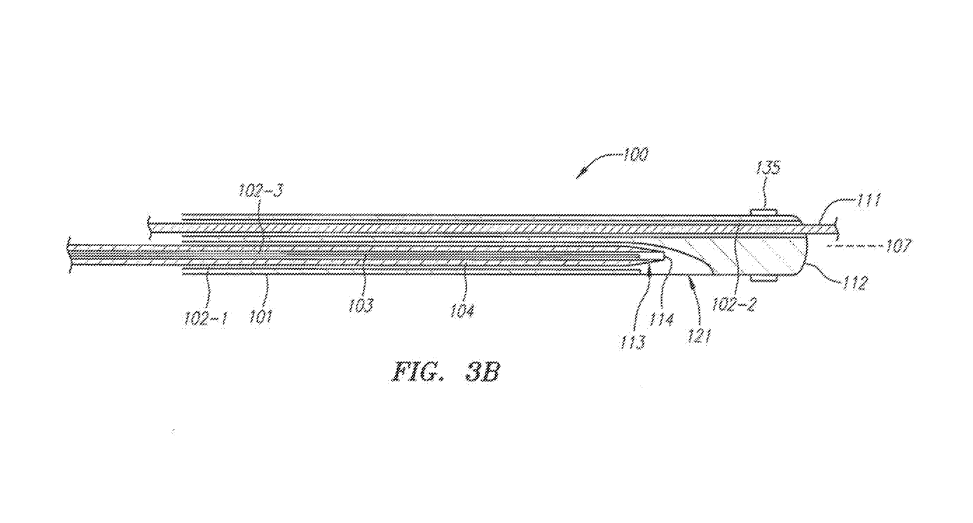

FIG. 3A is a perspective view depicting another exemplary embodiment of access system 100, which includes elongate member 101 having one or more lumens 102 (not shown), an elongate delivery device 104, and one or more open regions 121. Elongate member 101 is configured to house delivery device 104 within lumen 102-1 in a first, straight (or substantially straight) position, as shown in the longitudinal, cross-sectional, view of FIG 3B. Lumen 102-1 has exits from the sidewall of member 101, and has a distal curved section. As delivery device 104 is advanced from lumen 102-1, it contacts the curved wall of lumen 102-1 and is deflected, or re-directed, to a second position. Delivery device 104 exits open region (or port) 121 at a predetermined trajectory (or angle) offset fern longitudinal axis 107, preferably a trajectory chosen to position distal end 114 in proximity with fossa ovalis 208, as with the deployed second position depleted in FIG. 3A. Elongate member 101 and delivery device 104 may each include one or more lumens (e.g., 102-1 through 102-3), and each may optionally include an inter lumen exchange system 115 (not shown) to accommodate the presence of multiple inner lumens. Delivery device 104 here includes a tapered distal region 113 that can he used as a dilator. FIG. 3B also depicts optional guidewire 111. A guidewire 111 may be deployed through distal end 112, through open region 121, or through distal end 114 of delivery device 104.

Here, puncture device is housed directly within an inner lumen of delivery device 104. Alternatively, a tubular dilator 131 can be housed immediately within delivery device 104, with puncture device 103 housed immediately within tubular dilator 131. In this and the other embodiments described herein, elongate member 101 generally refers to the large diameter catheter while delivery device 104 generally refers to a smaller diameter catheter deployable from within elongate member 101 and. configured to deliver a treatment device (e.g., puncture device 103) or a diagnostic device.

An exemplary method for transseptal left atrium access using the exemplary embodiment of FIGS. 3A-B is provided. First, a patient's femoral vein or other veins capable of being used to access a right atrium 205 is accessed and a guidewire is touted through the access opening and into the right atrium, preferably from the IVC through the right atrium and into the SVC, in a manner similar to that described with respect to FIGS. 2A-F above.

Next, a proximal end of the guidewire 111 can be loaded into distal end 112 of elongate member 101. Elongate member 101 is then advanced along guidewire 111 into tight atrium 205, and preferably from the IVC 202 through right atrium 205 and into the SVC 203, such that a distal end of access system 100 is in the SVC 203. The distal position of elongate member 101 can be confirmed using an imaging technique. FIG. 3A shows radiopaque marker band 135 used for imaging with fluoroscopy. Next, distal end 114 of delivery device 104 is advanced, such as with proximal motion or through a proximal controller, from a first position, housed at least partially within elongate member 101, to a second position external to and deflected away from a longitudinal axis 107 of elongate member 101. Optionally, dilator 131 can be extended here. Next, access system 100 can be retracted and proximally rotated as necessary such that distal end 114 of delivery device 104 (or dilator 131) is just inferior of limbus 211. Next, the delivery device can be adjusted to a desired orientation with respect to a septal wall 207, such as septum primum 214, using an adjustment device 105 or proximal controller. FIG. 3A depicted system 100 in a desired position with delivery device 104 deployed against fossa ovalis 208. Next, using imaging techniques, the position of distal end 114 can be confirmed to be in a desired orientation with respect to septal wall 207, or the previous step may be repeated as necessary under imaging guidance until the appropriate amount of tenting, or dimpling of the fossa ovalis 208 is observed.

A hole can then be created in septal wall 207 with puncture device 103, such as by advancing a piercing stylet. Puncture device 103 preferable assumes as atraumatic shape after puncturing the patient's tissue and this can be confirmed using imaging techniques. If tapered, a distal region 113 of delivery device 104 can be advanced into the hole in the septal wall for dilation, and/or a separate dilator 131 can be advanced from distal end 114 into the hole. Then, puncture device 103 (see FIG. 3B) can be withdrawn from access system 100 and a left atrial guidewire can be advanced through access system 100 into left atrium 212. In systems that have a plurality of lumens, it may be possibly to advance a left atrium guidewire after retracting the puncture device into the delivery device, and not fully retracting it from the system. Once the left atrium guidewire is in position in the left atrium, the physician can withdraw the guidewire located in the SVC into the elongate member 101. If dilator 131 (not shown) was used it may then be retracted into delivery device 104. Finally, access system 100 may be removed leaving the left atrial guidewire in the left atrium.

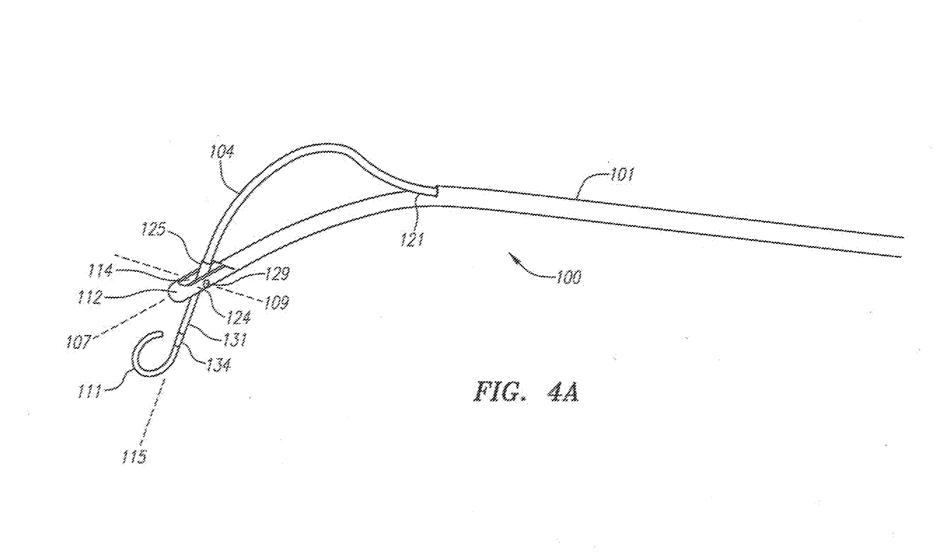

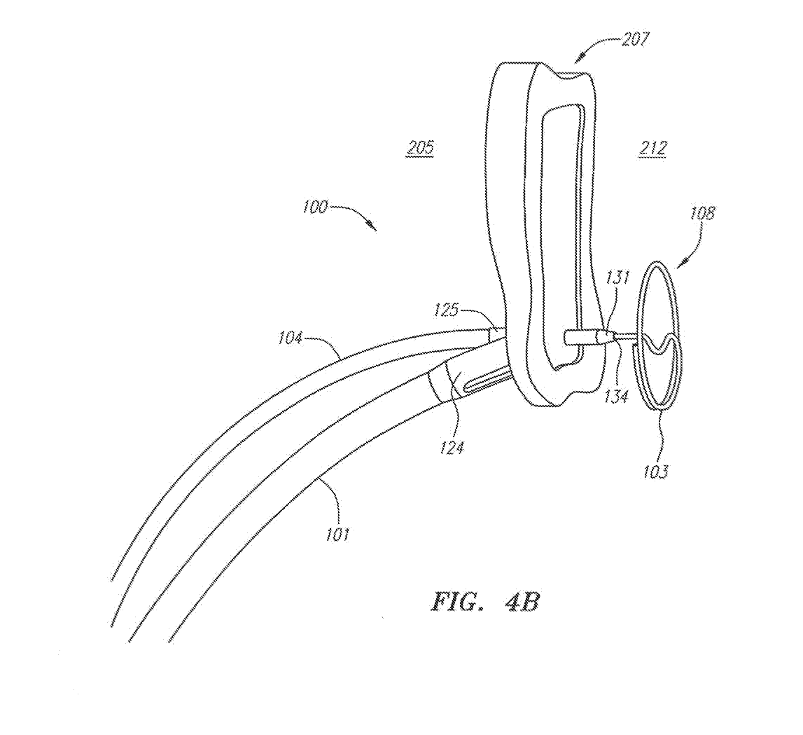

FIG. 4A is a perspective view depicting another exemplary embodiment of access system 100. In this embodiment, access system 100 comprises elongate member 101 with distal end 112, delivery device 104 with distal end 114, and a pull wire (not shown). Here, delivery device 104 is a second elongate member configured to be housed at least partially within elongate member 101. Delivery device 104 can be a hollow tubular member, including but not limited to, a catheter, typically with a diameter slightly less than elongate member 101. This facilitates the partial housing of delivery device 104 within elongate member 101, as shown here. In addition to a flexible tubular portion, delivery device 104 can include a rigid distal coupler 125 that is rotatably (or pivotally) coupled with elongate member 101. Delivery device 104 may be coupled such that distal end 114 of delivery device 104 can rotate latitudinally about a transverse axis 109 of elongate member 101. Here, delivery device 104 is coupled to member 101 with a swivel-type hinge 129, e.g., a rod that can rotate within rigid distal housing 124, which is a component of elongate member 101, or be affixed to housing 124 such that device 104 can rotate about the rod.

Delivery device 104 is housed within a lumen in member 101, and exits that lumen at port 121. The portion of delivery device 104 between coupler 125 and port 121 is exposed. During lateral positioning of distal end 114 of delivery device 104, advancement of delivery device 104 distally with respect to elongate member 101 causes this portion to are up or outwards, as illustrated in FIG. 4A. This position can likewise be accomplished by retracting elongate member 101 with respect to device 104 instead of, or in addition to distal advancement of delivery device 104. The longitudinal axis 115 at distal end 114 has been rotated from a first position directed (or facing) distally along longitudinal axis 107 of member 101 (e.g., as measured at distal end 112), to a second position offset from longitudinal axis 107 as shown here. The longitudinal axis 115 of delivery device 104 is now transverse to longitudinal axis 107 and no longer parallel (or approximately/generally parallel). Also shown here, is dilator 131 extended from within delivery device 104. Guidewire 111, in turn, is shown extended from within dilator 131. A puncture device 103 (such as a piercing stylet) can likewise be deployed from delivery device 104 or dilator 131. FIG. 4B depicts this embodiment in a deployed configuration with a piercing stylet 103 and dilator 131 deployed through the septal wall.

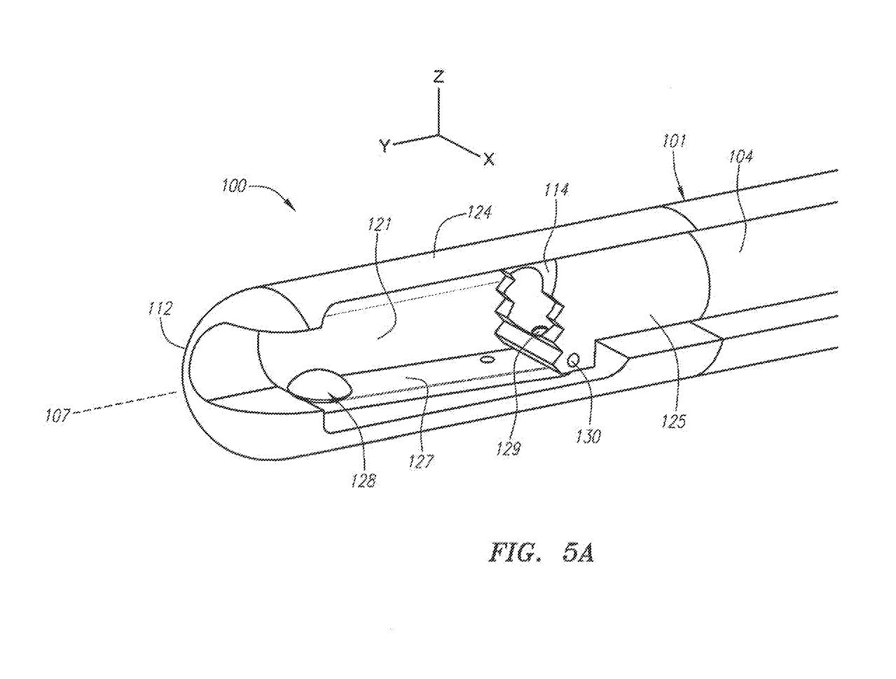

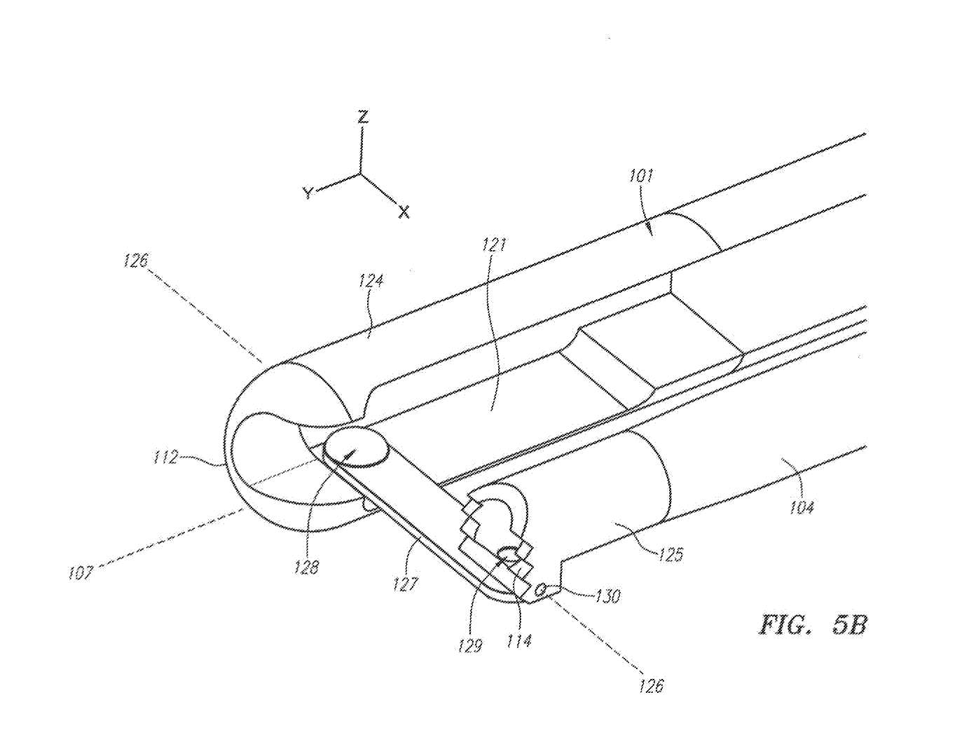

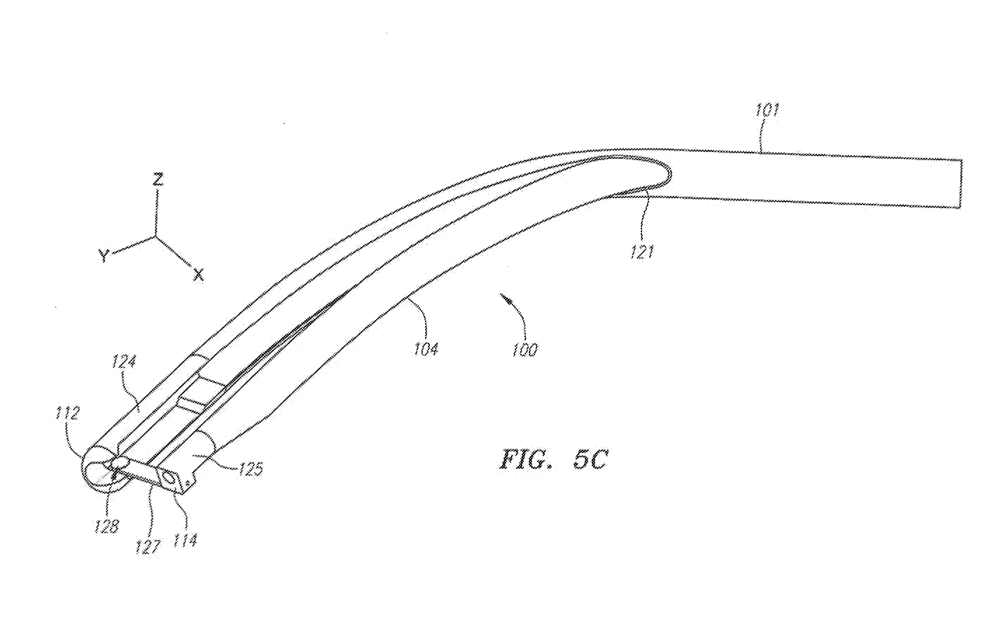

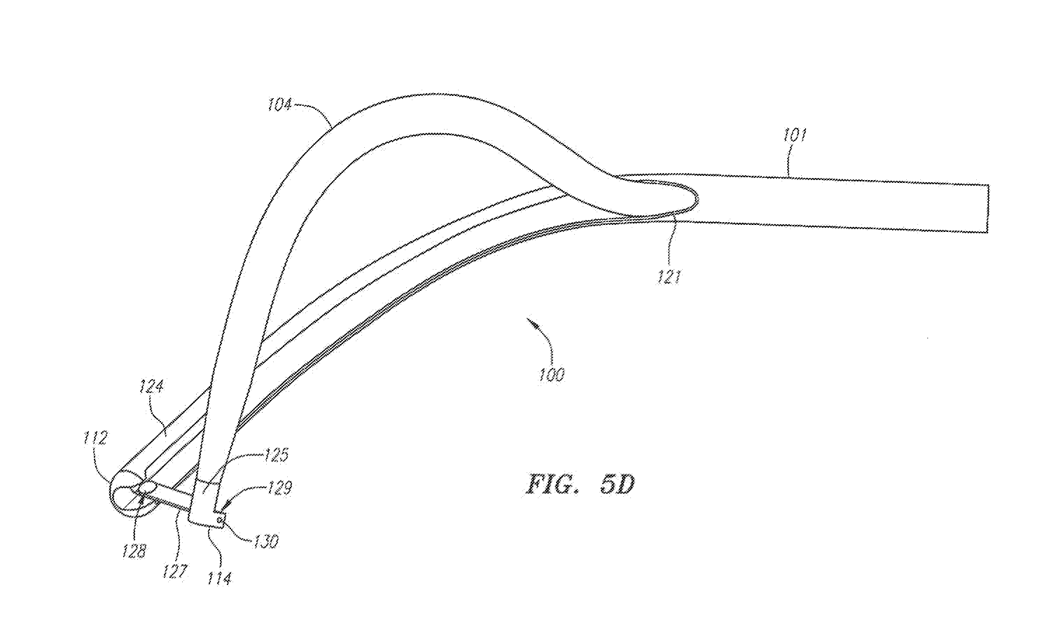

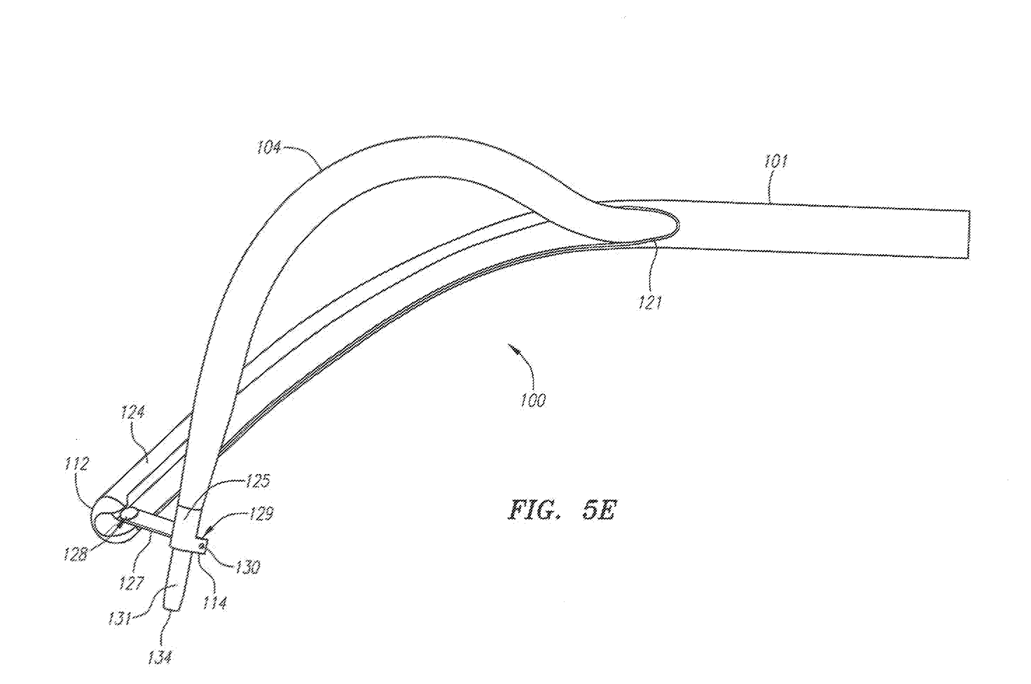

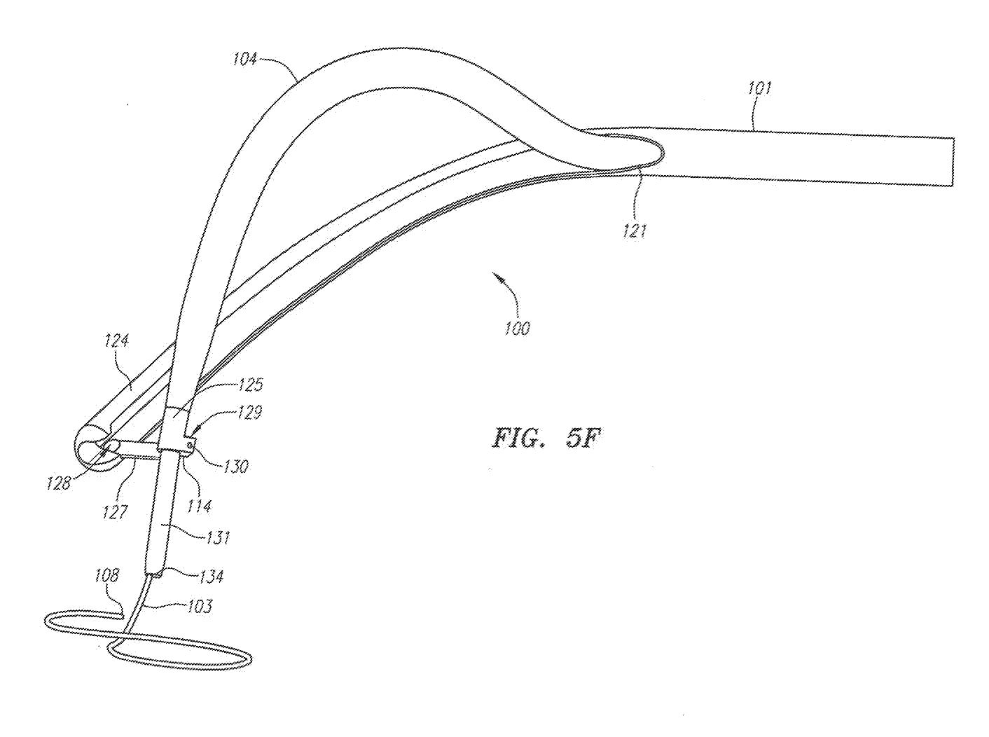

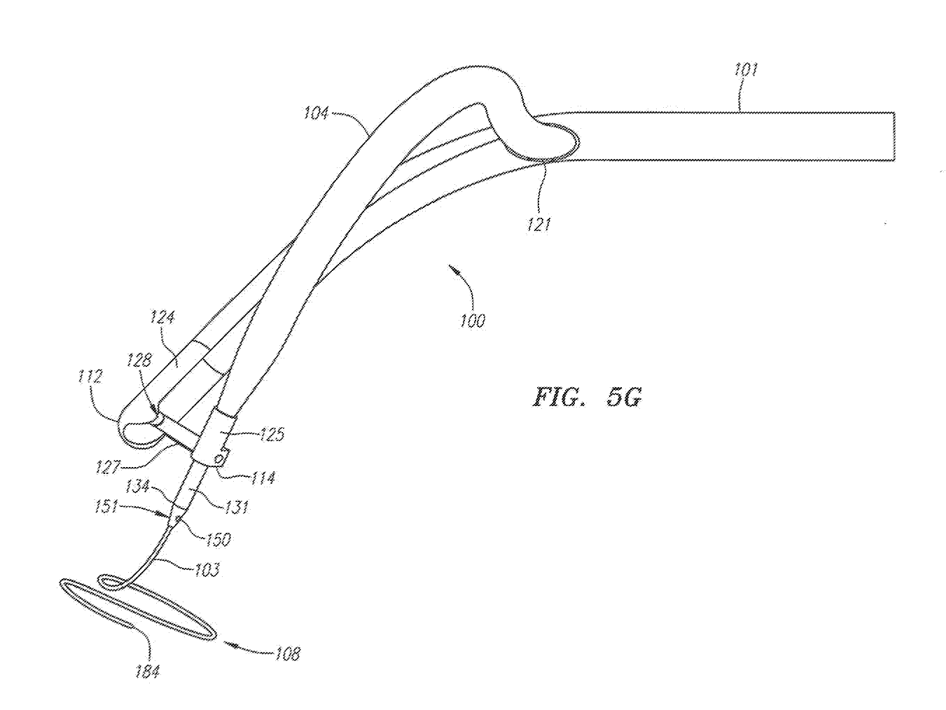

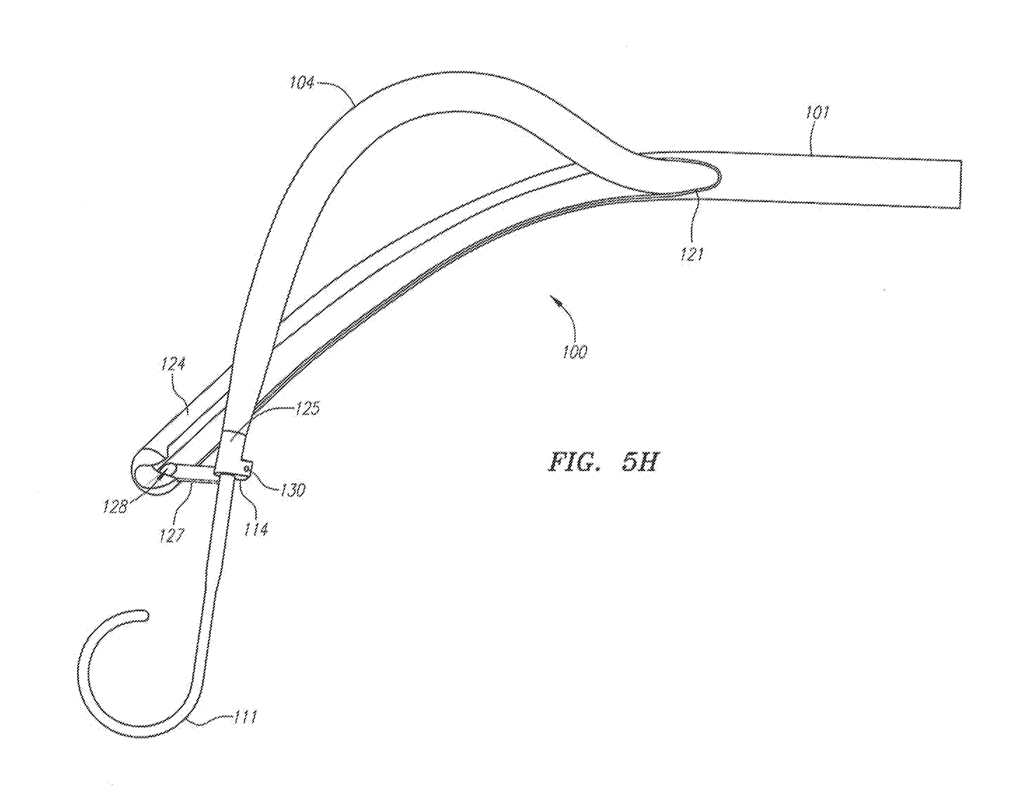

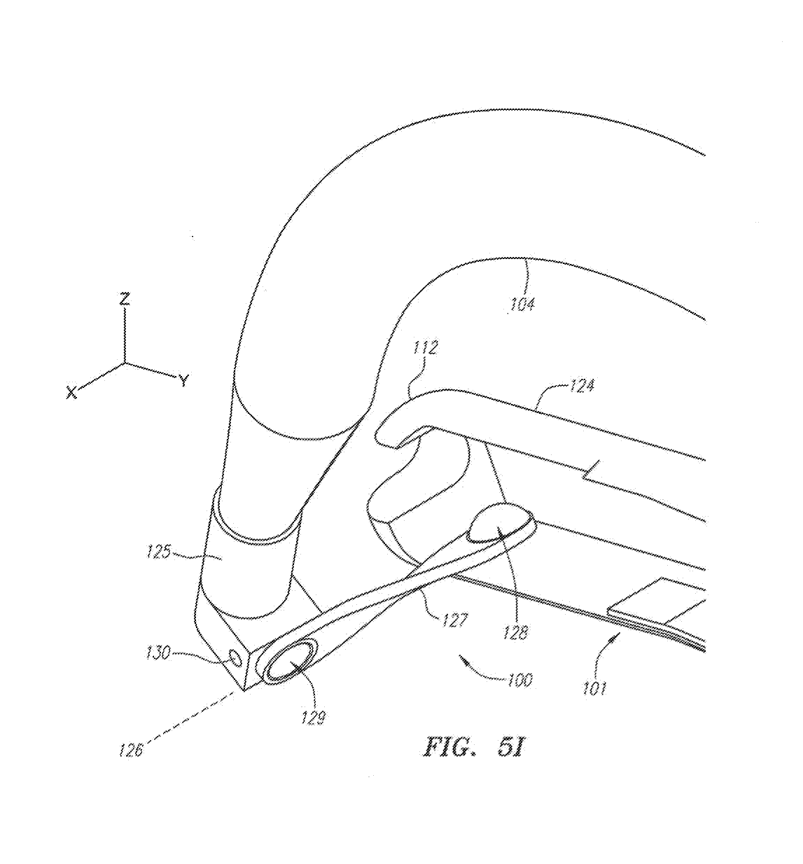

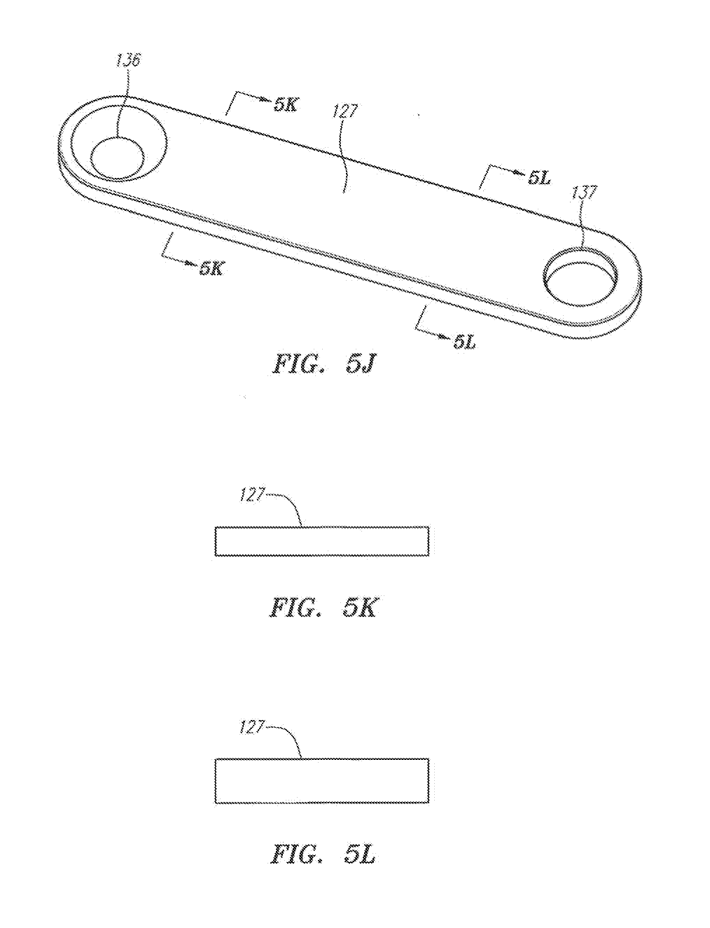

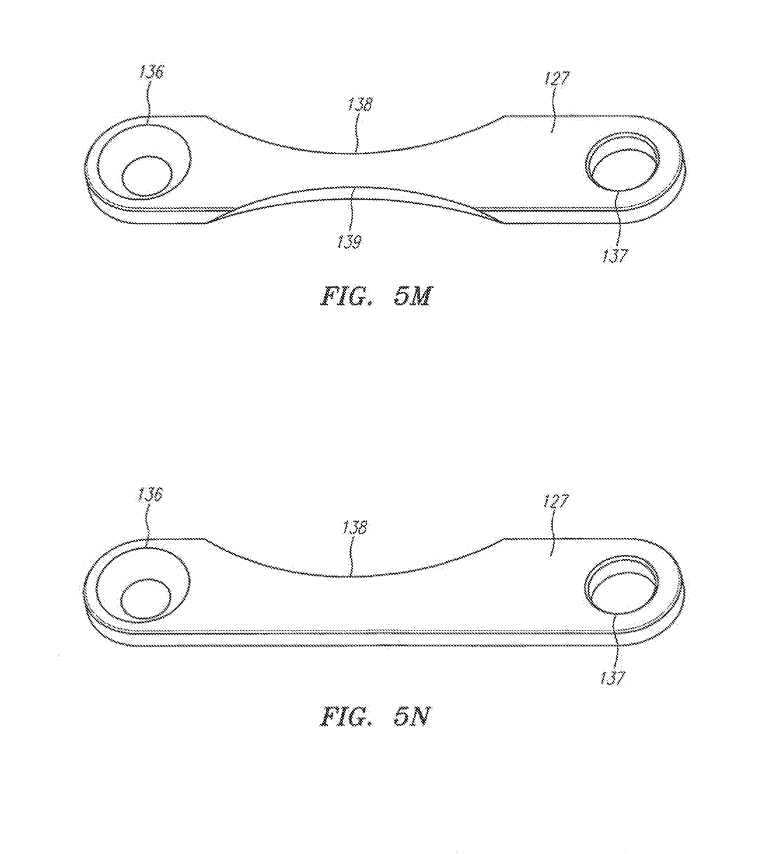

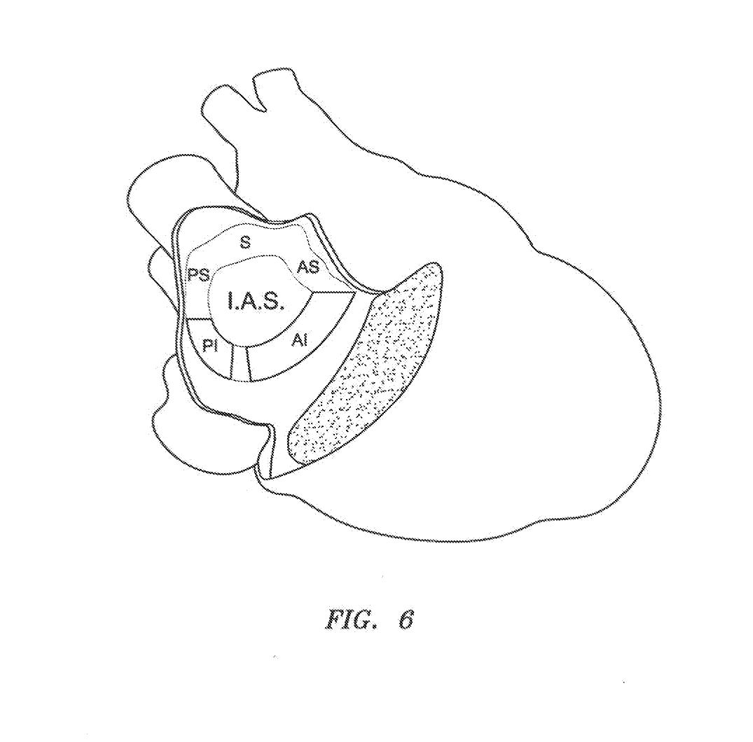

FIGS. 5A-O depict other exemplary embodiments similar to that described with respect to FIG. 4, but including an extension member 127. FIG. 5A is a side view depicting the distal portion of system 100 in a closed or housed, first position. Here, coupler 125 can be seen internal to elongate member 101 and with delivery device 104 seated within a channel in elongate member 101. Coupler 125 has multiple teeth to aid in engaging the septal tissue. Delivery device 104 is configured to deploy from within this channel with extension member 127 acting as a lever arm, as shown in the side view of FIG. 5B.

Extension member 127 is rotatably coupled (or pivotably) with elongate member 101 and with coupler 125. This could include any rotatable coupling including but not limited to a pin, pivot, swivel-type hinge, or living hinge. Extension member 127 is shown here after being swung outwards (in a car door like fashion) along the X-Y plane to a position approximately perpendicular to elongate member 101, although it should be noted that this position is variable and can be less than or more than perpendicular. Extension member 127 allows delivery device 104 to move from a first position housed at least partially within (or alongside, in the case there is no channel) elongate member 101 to a second position, such that distal end 114 of delivery device 104 is external to elongate member 101 in the second position.

Here, it can be seen that coupler 125 is rotatably coupled with extension, member 127 to provide two degrees of freedom. Extension member 127 is coupled to elongate member 101 with a swivel-type hinge 128 that allows extension member 127 to swing (or pivot) outwards. Hinge 128 is on a first end of extension member 127. On the second end of extension member 127 is hinge 129. Hinge 129 can be another swivel-type hinge and also allows delivery device 104 to swing (or pivot) laterally with respect to elongate member 101, again in the X-Y plane, while distal, end 114 can continue to point distally in a direction generally (i.e., not necessarily exactly) parallel to the longitudinal axis 107 of elongate member 101.

Coupler 125 can be coupled with hinge 129 in at least two different ways. First, coupler 125 can be coupled with hinge 129 by way of a second hinge 130. Hinge 130 is configured to allow delivery device 104 to swing (or pivot) in the Z-Y and Z-X planes (depending on the degree of rotation of extension member 127 in the X-Y plane). This allows the rotation of the longitudinal axis of delivery device 104 with respect to longitudinal axis 107 of elongate member 101. Although two hinges 129-130 are used to provide this freedom of motion, it should be understood that other adjustable or articulatable (e.g., ball and socket) hinges/connectors or flexible connections can be provided such as to accomplish the functionality of both hinges 129 and 130.

In an alternative embodiment, coupler 125 can be secured or fixed to hinge 129, such as by way of a static (non-rotating) coupling, such as a cross-pin, also depicted as numeral 130. This is shown in more detail in the perspective view of FIG. 5I. In this embodiment, extension member 127 is configured to flex (or twist), about its longitudinal axis 126, to allow delivery device 104 to swing (or pivot) along the X-axis in the Z-Y and Z-X planes.

Extension member 127 is preferably formed from an elastic or superelastic material, such as NITINOL. FIG. 5J depicts one exemplary embodiment of extension member 127. Here, member 127 has a first tapered aperture 136 to receive a flared edge of hinge 129 and hold member 129 to hinge 129 as shown in FIG. 5I. Extension member 127 also has a second aperture 137 to receive hinge 128, which is shown as a large-headed rivet at FIG. 5I. Extension member 127 can be configured with a variable cross-sectional thickness to decrease the force required to cause member 127 to flex or twist. FIGS. 5K and 5L are cross-sectional views taken along lines and 5K-5K and 5L-5L, respectively, to show how the cross-sectional thickness can be made to vary along the length of member 127.

FIGS. 5M and 5N are perspective views of additional exemplary embodiments of extension member 127 configured to flex in response to relatively less force, and also configured to reduce the stress applied to the material. Recessed, or cutaway, portions 138 and 139, are rounded surfaces that vary the width of member 127 and reduce the strain applied to member 127 during torsion. Recessed portions 138 and 139 can be present on one or both sides of member 127, as shown here. The reduction in width of member 127 provided by recessed portions 138 and 139 reduces the amount of material that must flex, and thereby reduces the force needed to twist. The rounded surface also more readily accommodates the strain profile that is incurred.

FIG. 5C is a perspective view depicting access system 100 after deployment of extension member 127, similar to FIG. 5B. Here, elongate member 101 is also known deflected downward. However, it could also be deflected left, right or upward. The deflection can be accomplished by use of a pull wire 105 (or other adjustment device) as described earlier, or elongate member 101 can be biased to deflect downward, such as through heat-treatment of the tubular body or the incorporation of wires that are pre-biased to deflect. In contrast to the embodiment described with respect to FIGS. 5A-B, the distal surface of coupler 125 is without teeth.

FIG. 5D depicts system 100 with delivery device 104 in the arced up (or away or out) position. This position, enables delivering the puncture device at the desired trajectory through the septal wall and can be accomplished by relative motion between elongate member 101 and delivery device 104, but preferably by advancing device 104 with respect to member 101. FIG. 5B depicts system 100 after an optional dilator 131 having a distal end 134 is partially extended from within delivery device 104. Here, dilator 131 is configured to be extended beyond or retracted within distal end 114 of delivery device 104. FIG. 5F depicts system 100 after puncture device 103 is partially deployed through distal end 134.

FIG. 5G depicts system 100 after the tapered section 151 of stylet 103 has been advanced from within dilator 131. (Proximal section 152 of stylet 103 is not visible within dilator 131.) Pressure sensor port 150 communicates with a pressure sensor lumen (not shown) that allows a fluid pressure measurement to be made at the proximal end of the system. It should be noted that an electronic or other pressure sensor can be used instead of the fluid pressure sensing port 150. FIG. 5H depicts system 100 after removal of dilator 131, pressure sensor device 151 and puncture device 103. A guidewire 111 is shown extending from delivery device 104.

Further, open region 121, delivery device 104, and extension member 127 can be configured so that delivery device 104 and extension member 127 can deploy up, down, left or right with respect to the deflection of elongate member 101.

An exemplary method for transseptal left atrium access capable of use with the exemplary embodiments of FIGS. 4A-5M is described. First, a patient's femoral vein or other veins capable of being used to access a right atrium 205 is accessed and a guidewire is routed through the access opening and into the right atrium (or past the right atrium and into the SVC), in a manner similar to that described with respect to FIGS. 2A-D above.

Next, a proximal end of a guidewire is loaded through distal end 112 of elongate member 101 and into a distal end 114 of delivery device 104. Member 101 is then advanced along the guidewire 111 into the right atrium 205, and preferably from the IVC through the right atrium and into the SVC, such that a distal end of access system 100 is in the SVC. The distal position of the elongate member 101 can be confirmed with an imaging technique such as fluoroscopy. Guidewire 111 can then be retracted through distal end 114 of delivery device 104 out of common lumen 117 and into a dedicated lumen 102. Next, access system 100 is retracted (proximally) from the SVC and positioned such that a distal end of the system is just inferior to the limbus. Preferably, system 100 is biased to curve towards the preferred location for puncture on the septal wall (e.g., via heat-treatment), allowing system 100 to be directed against the fossa ovalis. Alternatively, the adjustment device 105 (e.g., pull wire, etc.) is used to deflect access system 100 into the preferred location. This can be accomplished by deflecting elongate member 101 with adjustment device 105, in addition to any needed retraction, advancement or rotation of elongate member 101. Proper positioning is then confirmed with an imaging technique. Identification of the limbus and/or identification of any tenting, or dimpling, of the fossa ovalis can be used to help confirm proper positioning.

Next the orientation of distal end 114 of delivery device 104 is adjusted to a desired orientation with respect to septal wall 207. If an extension member 127 is included, this includes using the extension member 127 to position distal end 114 of the delivery device away from the elongate member 101 as shown in FIG. 5C (preferably prior to transitioning delivery device 104 to the arc-up position). Delivery device 104 can be transitioned to an arced position as shown in FIG. 5D, e.g., advancing device 104 from a first position, housed at least partially within elongate member 101, through open region 121 to a second position external to and deflected away from a longitudinal axis 107 of the elongate member 101 and in a desired orientation with respect to septal wall 207.

All embodiments may include a dilator 131. The dilator 131 may be extended (as shown in FIG. 5E) or retracted from elongate member 101 or delivery device 104. In some embodiments, the dilator 131 may be extended to show distension of the tissue. Distension of the tissue may be used to confirm positioning prior to creating a puncture, such as distension or “dimpling” of the septum primum 214, prior to performing a transseptal puncture or procedure. The dilator may be made of a variety of materials including but not limited to non-sharp plastics. The dilator may be used to expand a hole in a septal wall created after using puncture device 103, and to facilitate access of other medical devices across the hole. The dilator can also be used to form a slit instead of a hole. After the dilator has expanded the opening, a sheath might also be deployed from delivery device 104 to maintain the opening for later procedures.

Referring back to the method example, dilator 131 can then be optionally advanced from delivery device 104 into the fossa ovalis 208. Tenting, or dimpling, of the fossa ovalis 208 by dilator 131 can be confirmed through one of the aforementioned imaging techniques. A hole is then created in the septal wall 207. Typically this is accomplished using puncture device 103. Also, as described above the puncture device 103 typically assumes an atraumatic shape after puncturing the patient's tissue. (FIG. 5F shows the system 100 in this state with the tissue wall omitted.) The position of puncture device 103 can then be confirmed through one of the aforementioned imaging techniques. The physician (or other medical professional) can then perform a pressure measurement inside the left atrium to confirm the device position (see FIG. 5G). Next, a distal end of the access system 100 is advanced into the hole in the septal wall 207. This could include advancing dilator 131 or a sheath through the hole into the left atrium. Then, puncture device 103 can be withdrawn altogether from device 104. A left atrial access guidewire can then be loaded into the proximal end of device 104 (or, if already loaded in a guidewire lumen, then advanced distally from distal end 114) and advanced into the left atrium 212 through dilator 131.

Finally, dilator 131 can be retracted back into delivery device 104 (see FIG. 5H, again with the septal wall tissue omitted), and device 104 can be collapsed back to the configuration for housing within the elongate member 101 (and extension member 127, if present, can then be collapsed into elongate member 101). Access system 100 can then be removed altogether, leaving left atrial access guidewire 111 in a position extending through the atrial septal wall. Another medical device can be advanced over guidewire 111 and into the left atrium for performing a subsequent treatment or diagnostic procedure, either in the left atrium, left ventricle, or elsewhere within the heart.

Each embodiment preferably includes puncture device 103, or another apparatus for creating a hole in a patient's tissue. Puncture device 103 can comprise one or more of a variety of devices known in the art, including but not limited to solid or hollow needles, a Brockenbrough needle, a piercing stylet, or a sharpened guidewire. Alternatively puncture device 103 could use radio frequency ablation, optical energy, thermal energy, and the like. The puncture device 103 may be straight or curved, rigid or flexible, or may include a combination thereof. The puncture device 103 may be configured to be slidably housed at least partially within an inner lumen of the elongate member 101, delivery device 104, and/or within a dilator 131 attached to either elongate member 101 or delivery device 104.

Optionally, a distal portion of puncture device 103 may further be configured or biased such that it assumes an atraumatic shape upon puncturing the patient's tissue, such as after puncturing a septal wall during a transseptal crossing. The atraumatic shape is preferably configured such that it minimizes the risk of trauma to surrounding tissue, such as an aorta or left atrial far wall. A variety of shapes can be used.

In conventional systems, left heart access through the fossa ovalis is not performed in a relatively controlled or precise manner, resulting in punctures that are not in the preferred location. Puncture location has become increasingly more important through advancements made in structural heart interventions. FIG. 6 depicts the septal wall, with the fossa ovalis labeled as intra-atrial septum (IAS), with various puncture regions defined: superior (S); posterior-superior (PS); posterior-inferior (PI); anterior-superior (AS); and anterior-inferior (AI).

For example, patients that are treated for atrial fibrillation may have their left atrial appendage closed. Closure of the atrial appendage can be performed via a percutaneous access to the femoral vein and transseptal puncture. However, when performing transseptal puncture for closing the left atrial appendage (LAA), it is sometimes preferred to puncture the septum superiorly. It may be even more advantageous to puncture the septum on the posterior side as well as superiorly. A posterior-superior puncture aligns the treatment device for preferred access to the LAA. This puncture location may be considered safer as it would be further away from the aorta. The embodiments described with respect to FIGS. 5A-N are all configured for PS puncture.

A second example of a left heart interventional procedure that prefers a posterior superior puncture would be mitral valve edge-to-edge repair. In order to enter the valve in a perpendicular manner, the treatment device has to be properly aligned about the valve. The preferred alignment is achieved when the puncture location is performed in the PS position.

The physician may also desire to puncture the fossa ovalis in the anterior position. A device that facilitates anterior puncture in a safe manner would be advantageous.

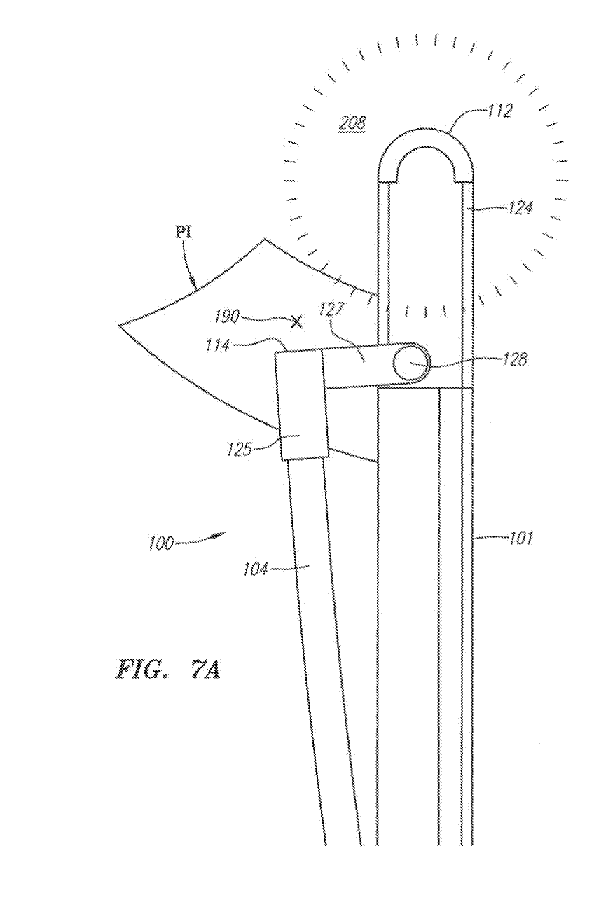

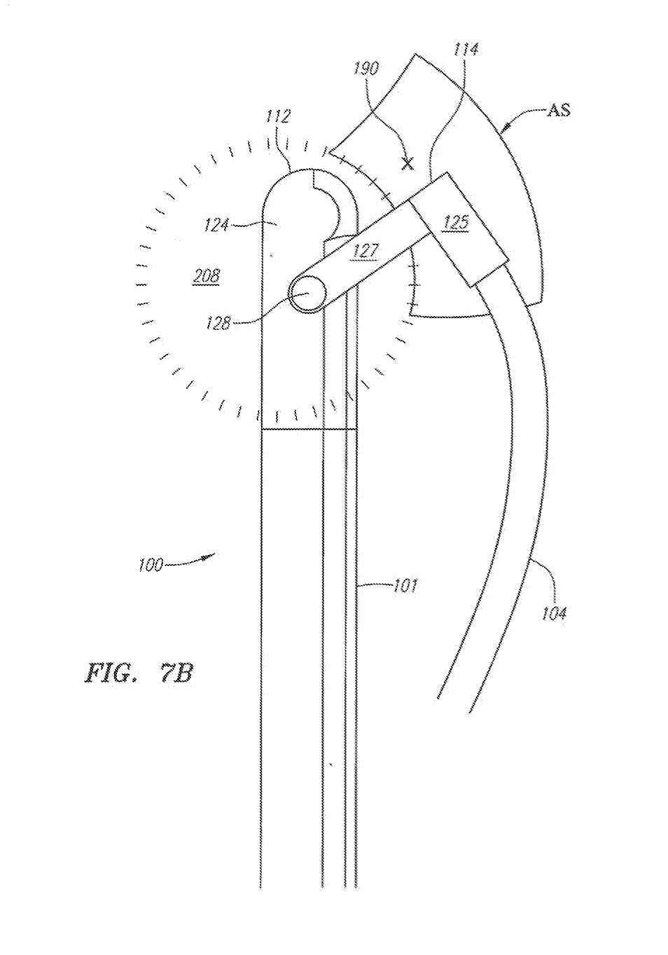

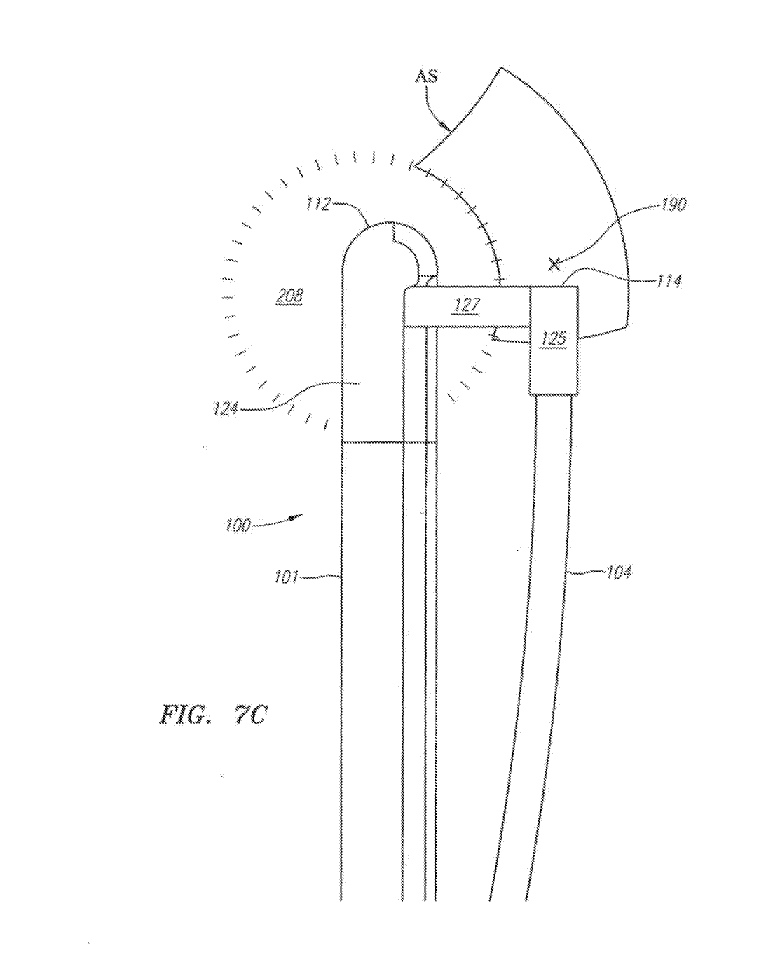

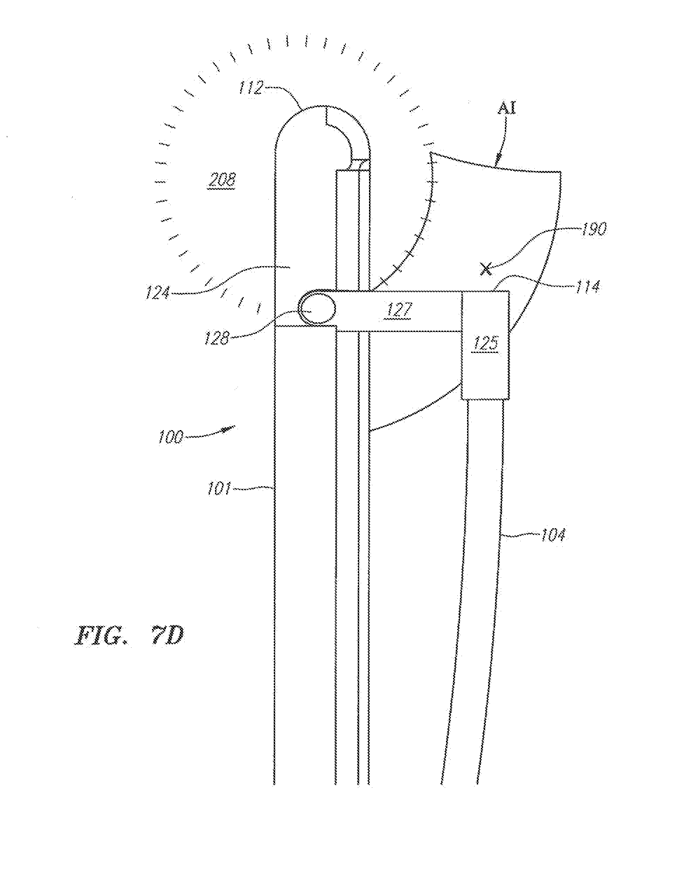

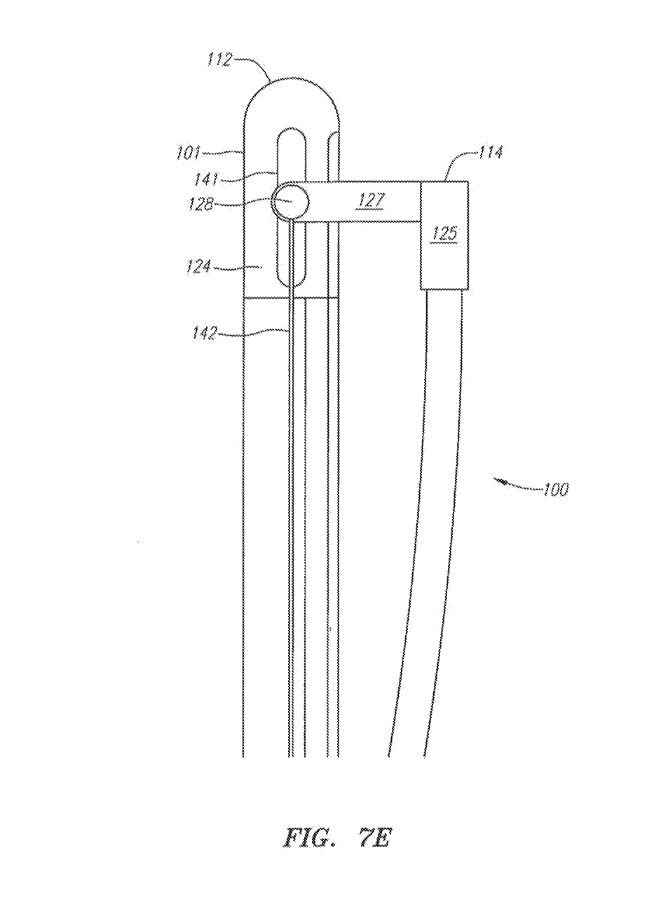

FIGS. 7A-E are top-down views of example embodiments of system 100 configured to puncture the septal wall at various locations. FIG. 7A depicts system 100 over fossa ovalis region 208. Here, delivery device 104 is shown extended from member 101 but not yet advanced into the arc-up position. Once advanced, distal end 114 will be over puncture location 190. Because extension arm 127 is configured to extend to the left side of member 101, and because the location of hinge 128 is located relatively further away from distal end 112 as compared to the embodiment of FIGS. 5A-H, puncture location 190 will be in the posterior-inferior (PI) region indicated. FIG. 7B shows another example where extension arm swings to the right side of member 101 and also swings distally to a one o'clock position, over the anterior-superior (AS) region indicated. FIG. 7C shows another example with a three o'clock AS puncture site 190. FIG. 7D depicts another example, where hinge 128 is placed relatively further back to allow a four o'clock anterior-inferior (AI) puncture site 190.

The distance from the main axis 107 of elongate member 101 (catheter body) is a variable that must be taken into consideration. For example, in a patient with a large fossa, it may be desirable to puncture 6 to 8 mm from the catheter axis and preferably 7 mm. In a patient with a small fossa it may be desirable to puncture at 4 to 5 mm. Achieving different puncture locations can be accomplished with different lengths of extension member 127, different degrees of allowable rotation and/or with a device where the pivot point is adjustable.

Due to the various of heart anatomy such as overall heart size, right and left atrial size, heart rotation and tissue thickness as well as histologic and pathologic conditions it may be advantageous to puncture the fossa in a variety of locations. For example, with a smaller heart that is rotated, it may be desired to puncture the septum primum posteriorly or inferiorly. The desired location for puncture location prior to the procedure and, therefore, may want to decide where to puncture once access to the right atrium is achieved.

FIG. 7E depicts an example embodiment that allows the position of extension member 127 to be adjusted. Here, hinge 128 engages and is slidable within a slot/track 141. The position of hinge 128 is controllable by an adjustment mechanism 143, that here takes the form of a push/pull wire. Push/pull 142 is coupled with hinge 128 and lies within a lumen internal to member 101. Pushing hinge 128 distally (or pulling hinge 128 proximally) adjusts the position of hinge 128 relative to distal end 112, so that the physician can adjust the puncture location between the superior position (just inferior to the limbus of the septum secundum), a mid-wall position or an inferior position at or near the base of the fossa ovalis. This can be done while member 101 remains in a static position, allowing the physician to adjust puncture location without adjusting position of the catheter itself.

Based on the description herein, it is possible to puncture the septal wall at one, two, three, four, five, seven, eight, nine, ten, eleven or twelve o'clock positions. For purposes herein, these positions are each defined as a 30 degree window (e.g., two o'clock is from 31-60 degrees about the central area of fossa ovalis 208, while ten o'clock is from 271-300 degrees about the central area). A two o'clock anterior superior position would be in the two o'clock position over the AS portion of the septal wall, which, unless specified otherwise, can he within the fossa ovalis or just outside the fossa ovalis.

Extension member 137 can also be configured to swing either to the left or the right of elongate member 101 as opposed to being fixed to deploy to only one of the two sides.

Turning back, to a method of used, although there are many different implementations and variations of method, for case of discussion, method will be described herein using an exemplary embodiment of access system 100 having puncture device 103, delivery device 104, and adjustment device 105.

The devices and methods herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within genitourinary tract in such areas as the bladder, urethra, ureters, and other areas.

One exemplary method for accessing a hollow organ other than the left atrium includes: accessing the organ percutaneously; advancing a guidewire 111 into the organ; advancing an elongate member 101 having a delivery device 104 along the guidewire 111 into the organ; optionally retracting the guidewire 111 at least partially into the delivery device 104 or elongate member 101; adjusting an orientation of the distal end of the delivery device 104 from a first position to a variable second position, wherein the second position is in a desired orientation with respect to a desired portion of the organ and is deflected away from the normal bias of a proximal end of the elongate member; and using the delivery device 104 to perform a desired medical procedure on the organ.

Furthermore, access system 100 may be used to pierce tissue and/or deliver medication, fillers, toxins, and the like in order to offer benefit to a patient. For instance, the device could be used to deliver bulking agent such as collagen, pyrolytic carbon beads, and/or various polymers to the urethra to treat urinary incontinence and other urologic conditions or to the lower esophagus/upper stomach to treat gastroesophageal reflux disease. Alternatively, the devices could be used to deliver drug or other agent to a preferred location or preferred depth within an organ. For example, various medications could be administered into the superficial or deeper areas of the esophagus to treat Barrett's esophagus, or into the heart to promote angiogenesis or myogenesis. Alternatively, the off-axis system can be useful in taking biopsies, both within the lumen and deep to the lumen. For example, the system could be used to take bronchoscopic biopsy specimens of lymph nodes that are located outside of the bronchial tree or flexible endoscopic biospy specimens that are located outside the gastrointestinal tract. The above list is not meant to limit the scope of the invention.

In some embodiments, access system 100 is used with an anchoring means in order to anchor the device to a location within the body prior to rotation of the off-axis system described with respect to FIGS. 4A-5N. This anchoring means may involve the use of a tissue grasper or forceps. It should be noted that any device or set of devices can be advanced within the lumen 102 of the access system 100, including but not limited to needles, biospy forceps, aspiration catheters, drug infusion devices, brushes, stents, balloon catheters, drainage catheters, and the like.

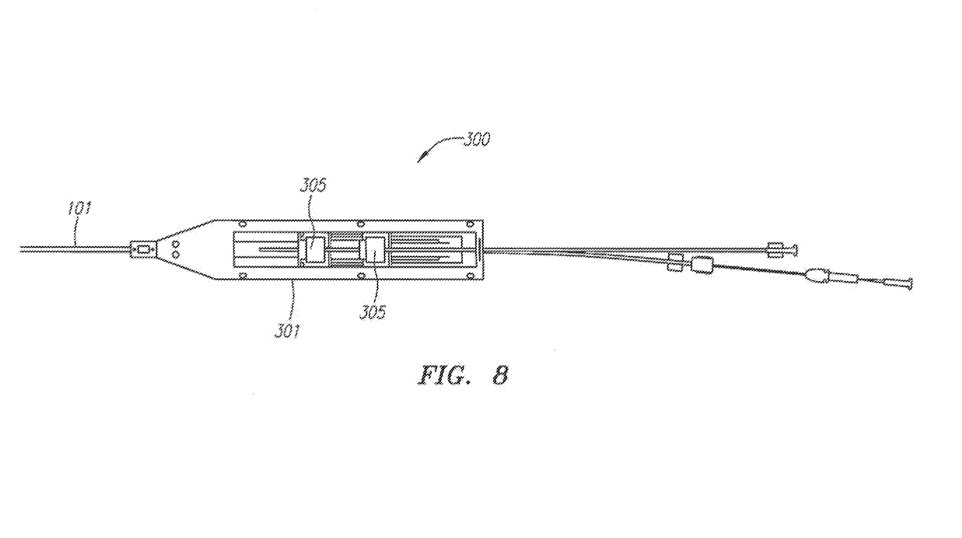

Control of access system 100 can be accomplished with the use of a proximal control device, or proximal controller. FIG. 8 depicts an exemplary embodiment of a proximal controller 300 including a handle 301 and a plurality of control tabs 305. The proximal controller is preferably configured to control each of the following (if present) adjustment device 105, delivery device 104, push/pull mechanism 143, dilator 131 and puncture device 103. The proximal controller can be configured to control the access system 100, the distal end of elongate member 101, and/or delivery device 104 along all three axes including left, right, up or down movement. The proximal controller can be configured in a manner similar to that described in Published U.S. Patent Application No. 2006/0112358, filed Jun. 29, 2006, and Published U.S. Patent Application No. 2008/0015633, filed May 4, 2007, both entitled Systems and Methods for Treating Septal Defects, and both of which are hereby incorporated by reference in their entirety for all purposes, and also specifically as they relate to the design and implementation of proximal controllers.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit of the disclosure. As used herein, the terms “exemplary” and “example” are interchangeable.

1. A method for transseptal left atrium access, comprising:
accessing a patient's vasculature percutaneously;
advancing an elongate member through the patient's vasculature and into the right atrium;
adjusting an orientation of the distal end of the elongate member from a first position to a variable second position, wherein the second position is in a desired orientation with respect to a septal wall and is deflected away from the longitudinal axis of the proximal end of the elongate member; and
creating a hole in the septal wall.

2. A method for transseptal left atrium access, comprising:
accessing a patient's vasculature percutaneously;
advancing a guidewire through the vasculature and into the right atrium; advancing an elongate member along the guidewire into the right atrium;
retracting the guidewire at least partially into the distal end of the elongate member;
adjusting an orientation of the distal end of the elongate member from a first position to a variable second position, wherein the second position is in a desired orientation with respect to a septal wall and is deflected away from the longitudinal axis of the proximal end of the elongate member;
creating a hole in the septal wall; and
advancing the distal end of the delivery device into the hole in the septal wall.

3. A method for transseptal left atrium access, comprising:
accessing a patient's vasculature percutaneously;
advancing a guidewire through the vasculature and into the right atrium;
advancing an elongate member, with an elongate delivery device, along the guidewire, wherein the elongate member includes an open region proximal to the distal end, and at least one inner lumen;
advancing the delivery device from a first position housed at least partially within the inner lumen of the elongate member to a variable second position, wherein the second position is deflected away from the longitudinal axis of the elongate member and is in a desired orientation with respect to a septal wall, further a distal end of the delivery device is extended through the open region of the elongate member in the second position;
creating a hole in the septal wall; and
advancing the distal end of the delivery device into the hole in the septal wall.

4.-13. (canceled)
